(19) United States
(12) Patent Application Publication
(10) Pub. No.: US 2004/0180850 A1
(43) Pub. Date: Sep. 16, 2004

(54) Methods and compositions for treatment of gastric diseases

(76) Inventors: Jari Natunen, Vantaa (FI); Halina Miller-Podraza, Vastra-Frolunda (SE); Susann Teneberg, Hindas (SE); Jonas Angstrom, Goteborg (SE); Karl-Anders Karlsson, Goteborg (SE)

Correspondence Address: BIRCH STEWART KOLASCH & BIRCH, PO BOX 747, FALLS CHURCH, VA, 22040-0747, US

(21) Appl. No.: 10/482,046

(22) PCT Filed: Jun. 28, 2002
(86) PCT No.: PCT/FI02/00575

(30) Foreign Application Priority Data
Jun. 29, 2001 (FI) 20011403
Jan. 18, 2002 (WO) PCT/FI02/00043

(51) Int. Cl. A61K 31/737; A61K 31/715

(52) U.S. Cl. 514/54; 514/55

(57) ABSTRACT

The present invention relates to a composition comprising a polysaccharide with Helicobacter pylori receptor activity and, optionally, an oligosaccharide receptor of Helicobacter pylori or an analogue or a derivative thereof and/or a gastric epithelium protecting compound for use in the treatment or prophylaxis of any condition due to the presence of Helicobacter pylori.

### FIELD OF THE INVENTION

The present invention describes polysaccharide substances useful for the treatment of gastric diseases. The polysaccharide substances are targetted to stabilize gastrointestinal mucus layer against ulcer causing agents and prevent the adhesion of Helicobacter pylori to oligosaccharide receptors in human stomach. The receptors are specific oligosaccharide sequences present on natural glycoconjugates such as glycoproteins and glycolipids. Specific embodiments describe the use of the polysaccharide substances together with receptor analogues and/or gastric pH regulating drugs.

### BACKGROUND OF THE INVENTION

The animal cells try to change the cell surface glycosylations, especially the terminal ones to prevent such bindings. The present invention shows a pathologic process where some terminal glycan parts are released by acid hydrolysis and receptors for Helicobacter pylori are formed. The present invention describes specific polysaccharide compositions which protect the gastric epithelium and mucosa from the chemical degradation and binding of the gastric pathogen Helicobacter pylori. The compositions preferably also comprise receptor analogues for Helicobacter pylori and/or gastric pH stabilizing drugs.

Several neutral and relatively rare oligosaccharide sequences are present on animal and human glycoconjugates. The saccharide sequences of asialo-GM1 (Galβ3GalNAcβ4Galβ4GlcβCer) and asialo-GM2 (GalNAcβ4Galβ4GlcβCer) glycosphingolipids are known receptors for numerous pathogenic agents including many pathogenic bacteria present in lungs, and viruses such as rotavirus.

US patents by Krivan, et al. U.S. Pat. No. 5,386,027 (January 1995) and U.S. Pat. No. 5,217,715 (June 1993) describe use of asialo-GM1 and asialo-GM2 to inhibit several pathogenic bacteria. The patent applications also suggest the pathogenic mechanism where desialylation by sialidase enzyme of influenza virus creates tne unsubstituted receptor GalNAcβ4Gal. However, in such structure GalNAc is not known to be sialylated and when Gal is α3-sialylated the structure is resistant to most sialidases by steric hindrance. Desialylation of other structures such as the terminal Gal of GM1b (NeuNAcα3 Galβ3GalNAcβ4Galβ4GlcβCer) is more likely to happen. The application also discuss an in vitro experiment where trachea of mouse is desialylated by 0.1 M hydrochloric acid and potential receptors for a lung pathogen is created. The application does not describe a natural condition in which such hydrolysis could happen nor methods to prevent it.

Lactocylceramide is also a common receptor for pathogenic bacteria, it has also been shown to bind cell surface polysaccharide present on pathogenic yeasts. Several viruses including human influenza virus, rotavirus, reovirus, mumps virus and rabies virus bind to glycolipids containing an oligosacharide sequence up to tetrasaccharide as exemplified by binding of sendai virus to lactosylceramide, GlcNAcβ3Galβ4Glcβ1Cer, GalNAcβ4Galβ4Glcβ1Cer, Galα3/4Galβ4Glcβ1Cer, Galβ3/4GlcNAcβ3Galβ4Glcβ1Cer (US patent Karlsson etal., U.S. Pat. No. 4,859,769, August 1989). US patent by Jimenez et al., U.S. Pat. No. 5,242,800 (September 1993) shows that lactosylceramide is receptor for pathogenic fungi. Oligosaccharide sequences Galα-, GalNAcαorβ-, or GlcNAcβ-3Galβ4GlcNAc are receptors for toxin A from Clostridium difficile (Teneberg et al., 1996). The saccharides GlcNAcβ3Galβ, Galβ3GlcNAc, Galβ3/4GlcNAcβ3Galβ4Glc, and Galβ4Glcβ are known as potential receptors for Streptococcus pneumoniae (Andersson et al., 1986). An accompanying patent application describes novel neutral oligosaccharide receptors for Helicobacter pylori which have saccharide sequence similar to the receptors of C. difficile. Helicobacter pylori is also known to bind lactosylcerarnide, asialo-GM1 and asialo-GM2-gangliosides. These studies do not relate to the use of polysaccharides.

Helicobacter pylori has been implicated in several diseases present in the gastrointestnal tract or other organs including chronic gastritis, non-steroidal anti-inflammatory drug (NSAID) associated gastric disease, duodenal and gastric ulcers, gastric MALT lymphoma and gastric adenocarcinoma (Axon, 1993; Blaser, 1992; DeCross and Marshall, 1993; Dooley, 1993; Dunn et al., 1997; Lin et al., 1993; Nomura and Stemmermann, 1993; Parsonnet et. al. 1994; Sung, et al., 2000; Wotherspoon et al., 1993). Totally or partially non-gastrointestinal diseases include sudden infant death syndrome (Kerr et al., 2000 and U.S. Pat. No. 6,083,756), autommune diseases such as autoimmune gastritis and pernicious anaemnia (Appelmelk et al., 1998; Chmiela et al, 1998; Clayes et al., 1998; Jassel et al., 1999; Steininger et al., 1998) and some skin diseases (Rebora et al., 1995), pancreatic disease (Correa et al., 1990), liver diseases including adenocarcinoma (Nilsson et al., 2000; Avenaud et al., 2000) and heart diseases such as atherosclerosis (Farsek et al., 2000). Multiple diseases caused or associated with Helicobacter pylorn has been reviewed (Pakodi et al., 2000). Of prime interest with respect to bacterial colonization and infection is the mechanism(s) by which this bacterium adheres to the epithelial cell surfaces of the gastric mucosa.

Glycoconjugates, both lipid- and protein-based, have been reported to serve as receptors for the binding of this microorganism as e.g. sialylated glycoconjugates (Evans et al., 1988), sulfatide and GM,3 (Saitoh et al., 1991), Le^{b} determinants (Borén et al., 1993), polyglycosylceramides (Miller-Podraza et al., 1996; 1997a), lactosylcernmide (Ångström et al., 1998) and gangliotetraosylceramide (Lingwood et al., 1992; Ångström et al., 1998). Another potential receptor for Helicobacter pylori is the phospholipid phosphatidylethanolarnine (Lingwood et al., 1992).

US patents of Zopf et al.: U.S. Pat. No. 5,883,079 (March 1999), U.S. Pat. No. 5,883,079 (March 1999), 5,753,630 (May 1998) and U.S. Pat. No. 5,514,660 (May, 1996) describe Neu5Acα6Gal-containing compounds as inhibitors H. pylori adhesion. Sialyl-factose molecule inhibits Helicobacter pylori binding to human gastrointestinal cell lines (Simon et al., 1999) and is also effective in an rhesus monkey animal model of the infection (Mysore et al., 2000). The compound is in clinical trials. The invention does not describe the conjugation sialylated saccharide with amine polysaccharide.

US patent Krivan et al. U.S. Pat. No. 5,446,681 (November 1995) describes bacterium receptor antibiotic conjugates comprising an asialo ganglioside coupled to a penicillin antibiotic. Especially is claimed the treatment of Helicobacter pylori with amoxicillin-asialo-GM1 conjugate. The invention is not related to the prevention of formation of neutral receptors or the use of polysaccharides.

Numerous drugs have been developed to treat gastric diseases. These include various proton pump inhibitor medicines such as omeprazole, molecules such as sucralfate with buffering capacity and possibly forming complex with gastric mucosa.

Chitosan-polysaccharide has been used for drug delivery to human stomach. The drug delivery form of chitosan is typically a hard compressed microsphere which is aimed at controlled release of a certain drug molecule in the desired conditions in gastrointestinal tract as described in U.S. Pat. No. 5,283,064 and 5,468,503. Present innovation describes the use of chitosan in a soluble or liquid form to achieve effective interaction with human mucus.

Some chitosan preparations have been described with potential positive effect on experimental gastritis in rat. Very large doses (best effect with 1 g/kg) of chitosan was needed because chitosan was not in a solubilized neutral form or suitable salt form (Ito, M. et al. 2000). Another study with suspension of insoluble and probably alkaline chitosan shows positive effect of chitosan against ulcers, molecular weight or origin of the preparation was not described (Hillyard, I. W. et al., 1964). The relevancy of the experimental rat disease to human conditions is not known.

Acidic polysaccharide dextran sulphate has been used to treat experimental Helicobacter pylori infection of mouse together with histamine H₂-receptor antagonist or proton pump inhibitor medicines (Icatlo, F. C. jr. et al., 2000). Relevancy of this treatment to human disease is not known. Dextran sulphate had also positive effect in a rat model when administered intravenously (Rudick, J., et al.,1968). U.S. Pat. No. 5,679,375 (1997) describes treatment of gastric and duodenal ulcers by high molecular weight sulphated polysaccharide. Carrageenan and sulphated amylopectin (SN-263, Depepsin) in a dog model (Ellis, C. M. et. al., 1970) and carragheen(carrageenan), chondroitin sulphate, heparin and dextran sulphate in a rat model (Barnes, W. k. et al., 1967) have anti-ulcers effects possibly depending on their protease inhibiting activities. The previous inventions do not describe combinations of acidic polysaccharides with amine polysaccharide for treatment of the gastric diseases. The present innovations relates to human specific gastric diseases, especially ones caused by the gastric pathogen Helicobacter pylori. This pathogen has adapted to live with human beings, it is a human specific pathogen, occassionally other primates or cats may be infected by some strains. It has several oligosaccharide binding specificities which can effectively recognize human type glycosylations relevant to gastric diseases and pathogenesis.

The novel combination therapies described are useful for treatment of human patients with several other gastric diseases, including gastric ulcers and duodenal ulcers even when Helicobacter pylori is not present. The oligosaccharide inhibitors are non-reactive molecules, which are present as natural components of human milk or as parts of human natural glycans. The oligosaccharide inhibitors are hydrophilic carbohydrates which are genarally not adsorbed at all or are adsorbed in only very small amounts to blood circulation from the gastrointestinal tract. The saccharide sequences can have several positive effects in protecting against other pathogens and stabilizing normal bacterial flora in gastrointestinal tract. There for these are safe to use and have positive effects in a general gastric therapy even when the presence of Helicobacter pylori is not demonstrated. According to present invention the receptor oligosaccharide sequences or their mimics are present in certain polysaccharides or polysaccharide complexes. The presence of receptor active sequences in polyvalent form makes the polysaccharides very useful for prevention of Helicobacter pylori Together with Helicobacter pylori or alone numerous agents are known to cause gastritis and gastric or duodenal ulcers such as non-steroidal anti-inflammatory drugs (NSAIDs) or alcohol. Alcohol induced gastric diseases have been discussed in U.S. Pat. No. 5,204,118, the negative effects of alcohol start from concentrations of about 10% of ethanol and 40% ethanol and higher concentrations are quite irritating and cause inflammation and can cause erosive gastritis.

Some polysaccharide compositions are known for the treatment of wounds in skin. The present invention describes use of similar compositions against gastric ulcers, especially in combination with inhibitors for Helicobacter pylori receptors and/or other gastric protective drugs.

### SUMMARY OF THE INVENTION

The present invention relates to a composition comprising a polysaccharide with Helicobacter pylori receptor activity and, optionally, an oligosaccharide receptor of Helicobacter pylori or an analogue or a derivative thereof and/or a gastric epithelium protecting compound for use in the treatment or prophylaxis of any condition due to the presence of Helicobacter pylori.

The present invention also relates to a repeating polysaccharide substance comprising a terminal oligosaccharide sequence according to formula

[Galβy]ₚHex(NAc)ᵣα/βzGalβ4Glc(NAc)ᵤ (II)

wherein p, r and u are each independently 0 or 1 and y is either linkage position 3 or 4, and z is either linkage position 3 or 4, and Hex is either Gal or Glc, so that when p is 1 and y=3, then Hex is Galβ or Glcβ and r=1, or p is 1 and y=4 then Hex is Glcβ and r=1, when p is 0, then z is 4, Hex is Gal and r is 1.

### BRIEF DESCRIPTION OF THE DRAWINGS

FIG. 1. EI/MS of permethylated oligosaccharides obtained from hexaglycosylceramide by endoglycoceramidase digestion. Gas chromatogram of the oligosaccharides (top) and EI/MS spectra of peaks A and B, respectively (bottom).

FIG. 2. Negative-ion FAB mass spectra of hexa-(A) and pentaglycosylceramide (B).

FIG. 3. Proton NMR spectra showing the anomeric region of the six-sugar glycolipid (A) and the five-sugar glycolipid (B). Spectra were acquired overnight to get good signal-to-noise for the minor type 1 component.

FIG. 4. Enzymatic degradation of rabbit thymus glycosphingolipids. Silica gel thin layer plates were developed in CM/H₂O, (60:35:8, by vol.). A and B, 4-methoxybenzaldehyde visualized plates. C, autoradiogram after overlay with ³⁵S-labeled Helicobacter pylori. 1, heptaglycosylceraride (structure 1, Table 1); 2, desialylated heptaglycosylceramide (obtained after acid treatmet); 3, desialylated heptaglycosylceramide treated with β4-galactosidase; 4, heptaglycosylceramide treated with sialidase and β4galactosidase; 5, reference glycosphingolipids from human erythrocytes (lactosylceramide, trihexosylceramide and globoside); 6, desialylated heptaglycosylceramide treated with β4-galactosidase and β-hexosaminidase; 7, heptaglycosylceramide treated with sialidase, β4-galactosidase and β-hexosaminidase.

FIG. 5. TLC of products obtained after partial acid hydrolysis of rabbit thymus heptaglycosylceramide (structure 1, Table I). Developing solvent was as for FIG. 4. A, 4-methoxybenzaldehyde-visualized plate; B, autoradiogram after overlay with ³⁵S-labeled Helicobacter pylori. 1, heptaglycosylceramide; 2, desialylated heptaglycosylceramide (acid treatment); 3, pentaglycosylceramide; 4, hydrolysate; 5, reference glycosphingolipids (as for FIG. 4).

FIG. 6. Dilution series of glycosphingolipids. The binding activity on TLC plates was determined using bacterial overlay technique. TLC developing solvent was as for FIG. 4. Different glycolipids were applied to the plates in equimolar amounts. Quantification of the glycolipids was based on hexose content. A, hexa- and pentaglycosylceramides (structures 2 and 3, Table I); B, penta- and tetraglycosylceramides (structures 4 and 5, Table I). The amounts of glycolipids (expressed as pmols) were as follows: 1, 1280 (of each); 2, 640; 3, 320; 4, 160; 5, 80; 6,40; 7, 20 pmols (of each).

FIG. 7. Thin-layer chromatogram with separated glycosphingolipids detected with anisaldehyde (A) and autoradiogram after binding of radiolabeled Helicobacter pylori strain 032 (B). The glycosphingolipids were separated on aluminum-backed silica gel60 HPTLC plates (Merck) using chloroform/methanol/water 60:35:8 (by volume) as solvent system. The binding assay was done as described in the “Materials and methods” section. Autoradiography was for 72 h. The lanes contained.

lane 1) Galβ4GlcNAcβ3Galβ4Glcβ1Cer (neolactotetraosylceramide), 4 μg;

lane 2) Galα3Galβ4GlcNAcβ3Galβ4GlcβCer (B5 glycosphingolipid), 4 μg;

lane 3) Galα3Galβ4GlcNH₂β3Galβ4Glcβ1Cer, 4 μg;

lane 4) Galα3(Fucα2)Galβ4GlcNAcβ3Galβ4Glcβ1Cer (B6 type 2 glycosphingolipid), 4 μg;

lane 5) GlcNAcβ3Galβ4GlcNAcβ3Galβ4Glcβ1Cer, 4 μg;

lane 6) Galβ4GlcNAcβ3Galβ4GlcNAcβ3Galβ4Glcβ1Cer, 4 μg;

lane 7) GalNAcβ3Galβ4GlcNAcβ3Galβ4Glcβ1Cer (x₂ glycosphingolipid), 4 μg;

lane 8) Neu5Acα3GalNAcβ3Galβ4GlcNAcβ3Galα4Glcβ1Cer (Neu5Ac-x₂), 4 μg;

lane 9) Fucα2Galβ4GlcNAcβ3Galβ4Glcβ1Cer (H5 type 2 glycosphingolipid), 4 μg;

lane 10) Neu5Acα3Galβ4GlcNAcβ3Galβ4Glcβ1Cer (sialylneolactotetraosylceramide), 4 μg. The sources of the glycosphingolipids are the same as given in Table 2.

FIG. 8. Thin-layer overlay assays to study the binding of Helicobacter pylori to polysaccharide materials (A) autoradiogram after binding of radiolabeled Helicobacter pylori strain 17875 (B). autoradiogram after binding of radiolabeled Helicobacter pylori strain 17874. The binding assay was done essentially as with glycolipids but the polysaccharides were just spotted on the on the plate and the molecules were not chromatographed with a solvent. Autoradiography was for 90 hours.

The spots contained:

spot A1) polysaccharide from Zooglea ramigera, 3 μg;

spot A2) chitosan, 3 μg;

spot A3) hyaluronic acid, 3 μg;

spot A4) chondroitin sulphate, 3 μg;

spot B5) polysaccharide from Zooglea ramigera, 0.6 μg;

spot B6) chitosan, 0.6 μg;

spot B7) hyaluronic acid, 1.5 μg;

spot B8) chondroitin sulphate, 0.6 μg;

spot C9) polysaccharide from Zooglea ramigera, 2 μg+chitosan, 2 μg;

spot C10) chitosan, 2 μg;

spot C11) hyaluronic acid, 2 μg+chitosan, 2 μg,

spot C12) chondroitin sulphate, 2 μg+chitosan, 2 μg,

spot D13) polysaccharide from Zooglea ramigera, 2)g+dendrimer, 2 μg;

spot D14) dendrimer, 2 μg;

spot D15) hyaluronic acid, 2 μg+dendrimer, 2 μg;

spot D16) chondroitin sulphate, 2 μg+dendrimer, 2 μg.

### DETAILED DESCRIPTION OF THE INVENTION

Present invention relates to oligosaccharide receptor sequences of animal and human cells which can serve as receptors for Helicobacter pylori. Many, if not most or all, pathogens bind to specific oligosaccharide sequences on animal cell surfaces. The present invention also teaches that neutral Helicobacter pylori receptor epitopes are revealed by acid hydrolysis during pathogenic conditions. The receptors formed are related to specially pathogenic conditions and therefore it is usefull to prevent formation of the receptors. The present invention describes specific polysaccharide compositions which protect the gastric epithelium and mucosa from the chemical degradation and binding of the gastric pathogen Helicobacter pylori. The compositions preferably also contain receptor analogues for Helicobacter pylori and/or gastric pH stabilizing drugs.

Helicobacter pylori Receptor Saccharide Sequences

Neutral, core type, receptor oligosaccharide sequences. A specific class of the Helicobacter pylori receptors are neutral and can be formed by acid hydrolysis of human or animal glycoconjugates. The neutral receptors are present in core regions of glycosphingolipids, in the backbone of common poly-N-acetyllactosamines or as defuicosylated forms of blood group A or B antigens. It is considered beneficial for a pathogen to target its binding to conserved parts of glycosylation. Many glycoconjugates contain acidic modifications by sialic acids or are fucosylated, these modifications are more terminal and varying, possibly due to evolutionary pressure by the pathogens. Such acidic saccharide sequences or fucosylated sequences and specific pathogen binding mechanisms to these are in the scope of a specific embodiment of the present invention. The fucose and sialic acid decoys are probably aimed by evolution to protect the core neutral glycan epitopes from binding of pathogenic agents, acid lability especially in stomach is a weak point of this protection.

Several methods to release sialic acids or N-acetylneuraminic acid and fucose residues from mammalian and human glycoconjutes are described in the Examples. The conditions are comparable to the strenght of gastric acid which may consist of 0.1 M hydrochloric acid. With even stronger acid treatment it is possible to release galactose and/or N-acetylglucosamine from the glycoconjugates. The authors used acid hydrolysis methods to create defucosylated and desialylated forms of glycolipids which where originally not Helicobacter pylori binding. Such neutral glycan receptors showed to be good receptors for Helicobacter pylori. Prevention of acidic conditions on gastric epithelium or neutralization of gastric acid prevents formation of such structures in a patient.

Under normal conditions in a healthy person a layer of mucin proteins is protecting the gastric epithelium and its glycoproteins and glycolipids from gastric acid which may correspond to 0.1 molar hydrochloric acid and very few of Helicobacter pylori reaches to contact with the epithelial cells, the bacteria live mainly in the mucin layer. pH on the gastric epithelium is close to neutral while pH in the gastric acid on the other side of the mucin layer can be 1-2. When pathogenesis starts the mucin layer gets thinner and weaker allowing gastric acid to leak to the epithelium and cause the formation of neutral oligosaccharide receptors for Helicobacter pylori. Other target cells such as granulocytes or lymphocytes can also be subjected to acid hydrolysis on weakly mucin protected epithelium.

The present invention also relates to a family of specific novel oligosaccharide sequences binding to Helicobacter pylori. These novel neutral oligosaccharide receptors, their analogs and use thereof are descnred in a co-pending application: Novel receptors for Helicobacter pylori and use thereof. The natural types of the receptors described including GlcNAcβ3Galβ4GlcNAc, GalNAcβ3Galβ4GlcNAc, GalNAcα3Galp4GlcNAc, Galβ3Galβ4GlcNAc, Galα3Galα4GlcNAc are especially of interest of present study as receptors made by degradation of larger oligosaccharide structures.

The receptors were characterized as glycolipids with sequences Galβ4GlcNAcα3Galβ4GlcβCer, Galβ4GlcNAcβ3Galβ4GlcNAcβ3Galβ4GlcβCer, GalNAcβ3Galβ4GlcNAcβ3Galβ4GlcβCer, GalNAcα3Galβ4GlcNAcβ3Galβ4Glc βCer, GlcNAcβ3Galβ4GlcNAcβ3Galβ4GlcβCer and Galα3Galβ4GlcNAcβ3Galβ4GlcβCer and Galα3Galβ4GlcNAcβ3Galβ4GlcNAcβ3Galβ4GlcβCer, but similar oligosaccharide sequences are also present on glycoproteins. The binding epitope was shown to include the terminal trisaccharide element of active pentasaccharide glycolipids, at least in larger repetitive N-acetyllactosamines the epitope may be also in the middle of the saccharide chain. The inventors realize that the binding epitopes can be presented in numerous ways on natural or biosynthetically produced glycoconjugates and oligosaccharide such as ones of O-linked or N-linked glycans of glycoproteins and on poly-N-acetyllactosamine oligosaccharides. The similarity of the epitopes was shown by molecular modelling of the glycolipids.

The Helicobacter pylori receptors which can be formed by acid hydrolysis in human stomach or gastrointestinal system also include asialo GM1, Galβ3GalNAcβ4Galβ4GlcβCer, asialo GM2, GalNAcβ4Galβ4GlcβCer, desialylated CAD epitopes such as GalNAcβ4Galβ4GlcNAcβ3Galβ4GlcβCer, and lactosylceramide, Galβ4Glcβ1Cer. Lactotetraosylceramide Galβ3GlcNAcβ3Galβ4GlcβCer is also a potential pathogenesis associated receptor for Helicobacter pylori. Desialylated CAD-epitopes and type 1 lactosamines, Galβ3GlcNAcβ3, are also present on glycoproteins after acid hydrolysis. It is realized that many other pathogenesis associated Helicobacter receptors can be formed from oligosaccharide chains of glycoproteins and glycolipids.

Collectively the essential lactose/lactosamine type receptor is an oligosaccharide sequence according to formula

[Galβy]ₚ[Hex(NAc)ᵣα/βz]ₛGalβ4Glc(NAc)ᵤ (I)

wherein p, r, s, and u are each independently 0 or 1, and y is either linkage position 3 or 4, and z is either linkage position 3 or 4, and Hex is either Gal or Glc, so that when p is 1, and y=3, Hex is Galβ or Glcβ and r=1, or p is 1 and y=4 and Hex is Glcβ and r=1 (the terminat Gal is β33- or β4-linked to GlcNAcβ or the terminal Gal is β3-linked to GalNAcβ), when p is 0 and z is 4, then Hex is Galβ and r is 1(the terminal monosaccharide structure is GalNAcβ4), or p=0 and z=3 (the terminal is HexNAc/Hexα/β3). Preferably the lactosamine/lactose type receptor activity is a neutral oligosaccharide sequence.

In another embodiment the lactosamine/lactose type receptor activity is a glycolipid type oligosacharide sequence, meaning that the types of Helicobacter pylori receptor oligosaccharide sequences belong to sequences present on animal glycospingolipids.

The other Helicobacter pylori receptors which can be used in covalent or non-covalent combinations with amine polysaccharides of the present invention are Lewis b saccharides, analogues thereof (Borén et al., 1993) and sialylated, especially NeuNAcα3Gal and NeuNAcα6Gal, Helicobacter pylori receptors (Evans et al., 1988; Miller-Podraza et al., 1996; 1997a).

Polysaccharides

The polysaccharide sequence used has preferably homology to the neutral receptor sequences. Even though the homology is not strong the polyvalency on the epitope will promote the contact of the polysaccharide with Helicobacter pylori. The glycolipid receptors contain for example lactosyl (Galβ4Glc) or N-acetyllactosaminyl (Galβ4GlcNAc, and GlcNAcp3Galp4GlcNAc also with terminal N-acetylglucosamine) sequences. Naturally identical or similar sequences are found from bacterial exopolysaccharides. For example the capsular polysaccharides of B-type Streptococcus contain α2-3sialylated variants and Streptococcus pneumoniae contains neutral variant of the receptor active sequence Galβ4GlcNAcβ3Galβ4Glc. Preferably the polysaccharide is produced by a non-pathogenic organism such as lacticacid bacteria. As an example of a large scale polysaccharide produced by a non-pathogenic bacterium and binding to Helicobacter pylori, we show the extracellular polysaccharide from Zooglea ramigera in examples. This polysaccharide has several epitopes similar to the neutral receptor described above, the polysaccharide has been reported to contain lactose epitopes with modifications on position 3 or 4 of Gal (Ikeda et al., 1982; Franzen and Norberg,1984). The polysaccharide chitosan has also similar structure with lactose(amine) sequences. The polymeric glucosamine has varying degree of acetylation, -GlcN(Ac)₀₋₁β4GlcN(Ac)₀₋₁β4GlcN(Ac)₀₋₁-with similarity to lactose, N-acetyllactosamine and GalNAcβGalβ4Glc/GlcNAc in some partially acetylated variants. Binding of Helicobacter pylori to chitosan and chitosan complexes with acidic polysaccharides has been also demonstrated in the examples.

The polysaccharide fucoidan has homology with facosylated Lewis type antigens such as Lewis b receptor of Helicobacter pylori and it is also sulphated.

Chondroitin/chondroitin sulphate and hyaluronic acid have homology with lactosamnine type polysaccharides. The present invention describes the use of the acidic polysaccharides as covalent or non-covalent complex with an amine polysaccharide such as chitosan.

To increase the homology between the polysaccharide receptor and receptor active oligosaccharide or polysaccharide, receptor active carbohydrate can be chemically conjugated to the polysaccharide backbone. The conjugation is preferably done directly by a stable chemical bond. Methods to produce amide bonded carbohydrates, reductively aminate oligosaccharides to polysaccharides and numerous other saccharide conjugation methods are known in the art. In a specific embodiment of the invention the oligosaccharide group is linked through a spacer molecule. In a preferred embodiment glycosylamnine chemnistry is used for the conjugation.

The use of chitosan against gastric ulcers in rats has been described (Ito, M. et al. 2000). However, the preparation has several problems: (1) It is administered in solution of 0.5% acetic acid miaking the preparation acidic which can cause damage to stomach. The chitosan acetic acid has capacity to bind free acid more than equivalent amount, giving it a possiblity to target the negative effect of the acid. (2) Due to low solubility and activity of the preparations, large amounts from 250 to 1000 mg/kg are required in substantial volume of water to produce the modest effects against gastric disease. Present innovation describes use of chitosan salts in neutral (pH between 6.5 and 7.5) and near neutral preparations having pH between about 6.0-6.5 or 7.5-8.0. In a specific embodiment a sligthly alkaline preparation low molecular weight preparation is used, such preparation have pH between 8.0-9.0, more preferably 8.0-8.5. The chitosan salts are also water soluble. In an other embodyment the chitosan salt preparation has an avarage molecular weight of 25 000 or less, prerentially between about 10 000-20 000, more preferably between 2000-10 000 or less. In another embodiment free chitosan oligosaccharides comprising mixture or separate oligosaccharides from disaccharide to about decasaccharide, more preferably between tetra- to about decasacccharide. Production of soluble and neutral chitosan salts has been described in U.S. Pat. Nos. 5,061,792 and 4,574,150 and in WO 8,707,618. Low molecular weight chitosans and chitosan oligosaccharides can be produced as known in the art by acid hydrolysis or chitosan degradating enzymes, for example.

In a specific embodiment other oligosaccharides or carbohydrates are linked to chitosan backbone, preferred are conjugates which have higher water solubility than the orginal chitosan molecule. Especially preferred are Helicobacter pylori receptor oligosaccharide sequences described above, most preferred are neutral Helicobacter pylori receptor oligosaccharides such as Galβ4GlcNAcβ3Galβ4Glc(NAc)₀₋₁, Galβ3GlcNAcβ3Galβ4Glc(NAc)₀₋₁, GlcNAcβ3Galβ4GlcNAc, GalNAcβ3Galβ4GlcNAc, GalNAcα3Galβ4GlcNAc, Galβ3Galβ4GlcNAc, Galα3Galβ4GlcNAc, Galβ4GlcNAc and analogues GlcNAcα/β3Galβ4GlcNAc, GlcNAcα3Galβ4GlcNAc, and lactose analogues of these which can be presented by formula [Hex(NAc)₀₋₁α/β3]₀₋₁Galβ4Glc(NAc)₀₋₁ as described in patent application FI 20010118; or lactotetraosylsaccharide Galβ3GlcNAcβ3Galβ4Glc or gangliotetraosylsaccharide Galβ3GalNAcβ4Galβ4Glc and derivatives and analogues thereof as described in PCT/SE/00/02567.

Beside the action against Helicobacter pylori the neutral or soluble forms of chitosan can be used to achieve effective interaction with human mucus. Such interaction is known to increase the amount of secreted mucus. Typically gastric diseases start with damage in the mucous layer, which protect the gastric epithelium from gastric acid, pathogens and degradating enzymes. The use of chitosan or other polysaccharides according to the invention have activity for restoring the mucous barrier. This is achieved by the increase of mucin secretion, by ionic cross-linking mucin glycoproteins to make stronger barrier and by buffer effect of the polysaccharides. An additional positive effect may be obtained using a polysaccharide with wound healing activity. In a specific embodiment another anionic polysaccharide is used instead of chitosan which is a β1-4-linked glucosamine polysaccharide, examples of anionic polysaccharides includes modified or covalently cross-inked chitosans or chitosan analogs containing amine-groups or other hexosamine polysaccharides such as polymer of a α1-4-linked galactosamme. The chitosan or chitosan oligosaccharide or analogue preparations thereof may contain only glucosamine or may be partially N-acetylatated.

Acidic polysaccharides especially desired to protect the gastric epithelium and having potential activity towards Helicobacter pylori includes glycosaminoglycans heparin, heparan sulphate, carrageenan, chondroitin, chondroitin sulphate, fucosylated chondroitin or chondroitin sulphate and hyaluronic acid, and similarily sulphated compounds such as heparinoids, dextran suphate, cellulose sulphate, starch sulphate, amylopectin sulphate, fucoidan or acidic/glucuronic acid containing bacterial polysaccharides such as Streptococcus pneumonia type m polysaccharide or glycosaminoglycan analogous polysaccharides found from bacteria such as specific stains of E. coli or Streptococcus. The acidic polysaccharides or their fragments can be used as covalently or non-covalently conjugated combinations with chitosan or analogous amine containing polysaccharide.

According to present invention it is especially useful to combine compounds preventing the actions of gastric acid against the gastric epithelium (Gastric pH stabilizing drug, which means compound capable stabilizing the pH on gastric epithelium to close natural near neutral pH) and polysaccharide-oligosaccharide-inhibitors against Helicobacter pylori. Some neutral oligosaccharide inhibitors against Helicobacter pylori has been described in patent applications FI 20010118 and PCT/SE/00/02567. In an specific embodiment the gastric pH stabilizing drug is a polysaccharide as desrcibed above and the polysaccharide is covalently conjugated with the neutral oligosaccharide inhibitor against Helicobacter pylori. The polyvalent conjugate binds efficiently to Helicobacter pylori which is secreted with polysaccharide or polysaccharide bound to soluble mucous material.

The target cells for Helicobacter pylori are primarily epithelial cells of the target tissue especially gastrointestinal tract. Glycosylation of the target tissue may change because of infection by a pathogen. Target cells may be also malignant, transformed or cancer/tumour cells in the target tissue. Transformed cells and tissues express altered types of glycosylation and may provide receptors to bacteria. Binding of lectins or saccharides (carbohydrate-carbohydrate interaction) to saccharides on glycoprotein or glycolipid receptors can activate cells, in case of cancer/malignant cells this may be lead to growth or metastasis of the cancer. Several of the oligosaccharide epitopes of the invention, such as GlcNAcβ3Galβ4GlcNAc (Hu, J. et al., 1994), Galα3Galβ4GlcNAc (Castronovo et al, 1989), and neutral polylactosamines (Stroud et al., 1996), have reported from malignant cells, to be cancer associated or cancer antigens. Helicobacter pylori is associated with gastric lyrnphoma. The sequence GlcNAcβ3Galβ4GlcNAc has also been described from deep gastric mucins by an antibody binding potential cancer associated structures (Hanisch F. -G. et al., 1993). The substances according to the invention can be used to prevent binding of Helicobacter pylori to premalignant or malignant cells and activation of cancer development or metastasis. Inhibition of the binding may cure gastric cancer, especially lymphoma.

Gastric pH regulating compound or gastric epithelium protecting compound means any drug capable to neutralize or to higher gastric pH or to protect the gastric epithelium from gastric acid. Gastric pH regulating drugs include special proton pump inhibitors such as omeprazole, esomeprazole, lansoprazole, rabeprazole, pantoprazole etc. and other pH regulating drugs such as histamine H2-receptor antagonists such as cimetidine, famotidine, or ranitidine, and other drugs with potential complexing or coating activities to form protecting layer on gastric epithelium such as sucralfte (sucrosepolysulphate aluminium salt), buffering salt compositions or inflammation reducing compounds which increase the gastric pH or the protecting mucosa on gastric epithelium (such as carbenoxolone). Especially useful gastric pH regulating drugs to be used according to the invention are molecules forming a layer protecting gastric epithelium from gastric pH, more preferably the gastric pH regulating drug is a carbohydrate, more preferably polysaccharide or oligosaccharide containing sulphate groups or carboxylic acid groups. The polysaccharide or analogue according to the invention or related to the polysaccharides to the invention have varying degree of binding activity towards gastric epithelium or residual mucin and are able to buffer against the gastric pH. Similarily in protection can be used a mixture of polysaccharides or cross-linked polysaccharides or polysaccharide and oligosaccharide.

Target cells also includes blood cells, especially leukocytes. It is known that Helicobacter pylori strains associated with peptic ulcer, as the strain mainly used here, stimulates an inflammatory response from granulocytes, even when the bacteria are non-opsonized (Rautelin et al., 1994a,b). The initial event in the phagocytosis of the bacterium most likely involves specific lectin-like interactions resulting the agglutination of the granulocytes (Ofek and Sharon, 1988). Subsequent to the phagocytotic event oxidative burst reactions occur which may be of consequence for the pathogenesis of Helicobacter pylori-associated diseases (Babior, 1978).

It is known that Helicobacter pylori can bind several kinds of oligosaccharide sequences. Some specificities of certain strains represent symbiotic interaction which does not lead to cancer or other severe conditions. The present data about binding to cancer-type saccharide epitopes indicates that the substance according to the invention can prevent pathologic interactions, and in doing this, it may not effect on some of the less pathogenic Helicobacter pylori bacteria/strains binding to other receptor sructures. Therefore total removal of the bacteria may not be necessary for the prevention of the diseases related to Helicobacter pylori. The less pathogenic bacteria may even have a probiotic effect as they can prevent the colonization of the pathogenic strains of Helicobacter pylori in the gastric tract.

In one embodiment of the invention it is possible to incorporate the substance according to the invention to a carrier for use in a pharmaceutical composition, which is suitable for the treatment of a condition due to gastric disease, especially due to the presence of Helicobacter pylori in the gastrointestinal tract of a patient. It is also possible to use the substance according to the invention in a method for the treatment of such conditions. Examples of conditions treatable according to the invention are chronic superficial gastritis, gastric ulcer, duodenal ulcer, non-Hodgkin lymphoma in human stomach, gastric adenocarcinoma, certain pancreatic, skin, liver, or heart diseases, sudden infant death syndrome, autoimmune diseases including autoimmune gastritis and pernicious anaemia and non-steroidal anti-inflammatory drug (NSAID) related gastric disease involving also Helicobacter pylori.

The polysaccharide compositions have gastric epithelium protecting activities which make these useful also for therapy of gastric diseases such as gastritis, chronic superficial gastritis, gastric ulcer, duodenal ulcer, non-steroid anti-inflammatory drug (NSAID) related gastric disease, and alcohol induced gastric diseases such as gastritis, gastric ulcer or duodenal ulcer even when Helicobacter pylori is not present.

It is especially useful to use other gastric pH regulating drugs together with polysaccharides and/or receptor oligosaccharides according to the invention. In such therapy the formation of the pathogenesis associated receptors is hindered and binding to residual receptors formed is prevented by antiadhesive carbohydrates or analogues or derivatives thereof Some antiadliesive receptor oligosaccharides and derivatives and analogues thereof has been described in a related patent application titled Novel receptors for Helicobacter pylori and use thereof (FI20010118). The gastric pH-regulating drugs have side effects in elongated use. When such drugs are used together with the anti-adhesive polysaccharides or polysaccharide conjugates, the amounts of the drugs needed are lower and side effects weaker. When pathogenic bacteria diminish due to the lack of receptors, the patient can be cured without removal of all the harmless types of Helicobacter pylori. The gastric pH-regulating drugs and oligosaccharide receptors or analogues can also be used together with one or several, typically two or three different antibiotics such as amoxicillin, clarithromycin, metronidazole or rifabutin or bismuth compounds, to eradictate Helicobacter pylori in severe diseases such as in the case of gastric lymphoma.

The pharmaceutical composition according to the invention may also comprise other substances, such as an inert vehicle, or pharmaceutically acceptable carriers, preservatives etc, which are well known to persons skilled in the art.

The substance or pharmaceutical composition according to the invention may be administered in any suitable way, although an oral administration is preferred.

In a preferred pharmaceutical composition the Helicobacter pylori receptor activity is present on a polysaccharide or a modified polysaccharide derived from a bacterium or another microorganism. More preferably the polysaccharide is derived from a bacterium which is non-pathogenenic to human.

In another pharmaceutical composition the Helicobacter pylori receptor activity is present on a modified polysaccharide and the receptor active oligosaccharide sequence is chemically conjugated to a polysaccharide. More preferably a lactose(amine) oligosaccharide sequence is chemically conjugated to amine containing polysaccharide. In another preferred embodiment an acidic receptor polysaccharide or a fragment of an acidic polysaccharide receptor for Helicobacter pylori is chemically conjugated to amine containing polysaccharide, such as chitosan.

It is noted that many polysaccharides, especially bacterial polysaccharides, contain saccharide sequences which can be modified by a glycosyltransferning enzyme. Monosaccharide or several monosaccharides on the polysaccharides can be transferred so that the receptor active oligosaccharide sequence is formed on the polysaccharide. The glycosyltransferring enzyme can be a glycosidase, glycosyl transferase or transglycosylating enzyme. In a preferred embodiment terminal acceptor sequences are modified by glycosyltransferase or transglycosylating enzymes. Especially following reactions are preferred: 1) a terminal Glc is modified by β4-galactosyltransferase 2) a terminal GlcNAc residue is modified by β3- or β4-galactosyltransferase, 3) a terminal Lac or LacNAc residue is modified by β3-N-acetylglucosaninyltransfemase, β3-N-acetylgalactosaminyltransferase, β4-N-acetylgalactosaminyltransferase, or α3-galactosyltransferase, and 4) terminal GalNAcβ4Gal is modified by β3-galactosyltransferase.

Another preferred embodiment of the invention is a repeating polysaccharide substance comprising several of the following terminal oligosaccharide sequences according to formula

[Galβy]ₚHex(NAc)ᵣα/βzGalβ4Glc(NAc)ᵤ (II)

wherein p, r, and u are each independently 0 or 1 and y is either linkage position 3 or 4, and z is either linkage position 3 or 4, and Hex is either Gal or Glc, so that when p is 1, and y=3, Hex is Galβ or Glcβ and r=1, or p is 1 and y=4 and Hex is Glcβ and r=1, when p is 0, then z is 4, Hex is Gal and r is 1, or z=3.

The repeating polysaccharide substance refers especially to microbial, bacterial or other repeating polysaccharide which may have been modified to contain the terminal oligosaccharide sequence. The terminal sequence means that the monosaccharide residues are not modified by other monosaccharide residues except that at the reducing end. Preferably the repeating polysaccharide has molecular weight of >2000 Da, more preferably >10 000 Da.

The methods of treatment or pharmaceutical compositions utiizig specific pathogenesis associated oligosaccharide receptors are especially usefuil against pathogenic strains of Helicobacter pylori.

A preferred food-stuff of the invention is an infant formula. A preferred beverage is an alcoholic beverage comprising at least 10% ethanol and more preferably at least 30% ethanol. The polysaccharide compositions according to the invention can be used also in pharmaceuticals or feedstuffs for animals which can be infected by Helicobacter pylori, such as pigs or cats, for example. The compositions comprising carbohydrates as described by invention are preferred. The compositions have positive effect on the gastric health of an animal even when Helicobacter pylori or the like are not present by protecting the gastric epithelium.

Polysaccharide compositions described by the invention can be used in pharmaceutical compositions to prevent harmfull side effects of other drugs in stomach. For example, gastric irritation, gastritis and gastric ulcers can be caused by common drugs such as aspirin. Preferably the drug belongs to non-steroidal anti-inflammatory drugs (NSAIDs).

The term “treatment” used herein relates to both treatment in order to cure or alleviate a disease or a condition, and to treatment in order to prevent the development of a disease or a condition. The treatment may be either performed in a acute or in a chronic way. The glycosidase inhibitors can be administered together with other drugs such as known antibiotics used against the bacteria, virus, or fungus being the pathogenic agent.

The term “patient”, as used herein, relates to any human or non-human mammal in need of treatment according to the invention.

Furthermore, it is possible to use the substance according to the invention as a part of a nutritional compositon, for example in food or beverage composition. It is preferred to use the substance according to invention as a part of a so called functional or functionalized food. The said functional food has a positive effect on the person's or animal's health by inhibiting or preventing the binding of Helicobacter pylori to target cells or tissues. The substance according to the invention can be a part of definied food or functional food composition. The functional food can contain other known food ingredients accepted by authorities controlling food such as Food and Drug Administration in USA. The substance according to invention can also be used as a food additive, preferably as a food additive to produce a functional food.

Glycolipid and carbohydrate nomenclature is according to recommendations by the IUPAC-IUB Commision on Biochemical Nomenclature (Carbohydrate Res. 1998, 312, 167; Carbohydrate Res. 1997, 297, 1; Eur. J. Biochem. 1998, 257, 29).

It is assumed that Gal, Glc, GlcNAc, and Neu5Ac are of the D-configuration, Fuc of the L-configuration, and all the monosaccharide units in the pyranose form. Glucosamine is referred as GlcN or GlcNH₂ and galactosamine as GalN or GalNH₂. Glycosidic linkages are shown partly in a shorter and partly in a longer nomenclature, the linkages of the Neu5Ac-residues α3 and α6 mean the same as α2-3 and α2-6, respectively, and with other monosaccharide residues α1-3, β1-3, β1-4, and β1-6 can be shortened as α3, β3, β4, and β6, respectively. Lactosamine refers to N-acetyllactosamine, Galβ4GlcNAc, and sialic acid is N-acetylneuraminic acid (Neu5Ac) or N-glycolylneura inic acid (Neu5Gc) or any other natural sialic acid. Term glycan means here broadly oligosaccharide or polysaccharide chains present in human or animal glycoconjugates, especially on glycolipids or glycoproteins. In the shorthand nomenclature for fatty acids and bases, the number before the colon refers to the carbon chain lenght and the number after the colon gives the total number of double bonds in the hydrocarbon chain.

The inventors have found out that oligosaccharide sequences containing glucuronic acid and some derivatives thereof bind Helicobacter pylori. The new binding oligosaccharide sequences include sequence GlcAβ3Galβ4GlcNAcβ3Galβ4Glc. One embodiment of the present invention is the use of a substance or a receptor binding to Helicobacter pylori comprising the oligosaccharide sequence

[Gal(A)_{q}(NAc)ᵣ/Glc(A)_{q}(NAc)ᵣα3/β3]ₛ[Galβ4GlcNAcβ3]ₜGalβ4Glc(NAc)ᵤ

wherein q, r, s, t, and u are each independently 0 or 1,

so that when t=0 and u=0, then the oligosaccharide sequence is linked to a polyvalent carrier or present as a free oligosaccharide in high concentration, and analogs or derivatives of said oligosaccharide sequence having binding activity to Helicobacter pylori for the production of a composition having Helicobacter pylori binding or inhibiting activity.

A in the above oligosaccharide sequence indicates uronic acid of the monosaccharide residue or carbon 6 derivative of the monosaccharide residue, most preferably the derivative of carbon 6 is an amide of the uronic acid.

The Helicobacter pylori binding oligosaccharide sequence has been reported in the structure GlcNAcβ3Galβ4GlcNAcβ6GalNAc from human gastric mucins. This mucin epitope and similar O-glycan glycoforms are most probably natural high affinity receptors for Helicobacter pylori in human stomach This was also indicated by high affinity binding of an analogous sequence GlcNAcβ3Galβ4GlcNAcβ6GlcNAc as neoglycolipid to Helicobacter pylori and that the sequence GlcNAcβ3Galβ4GlcNAcβ6Gal also has some binding activity towards Helicobacter pylori in the same assay. Therefore the preferred oligosaccharide sequences include O-glycans and analogues of O-glycan sequences such as GlcNAcβ3Galβ4GlcNAcβ6GlcNAc/GalNAc/Gal, GleNAcβ3Galβ4GlcNAcβ6GlcNAc/GalNAc/GalαSer/Thr, GlcNAcβ3Galβ4GlcNAcβ6(Gal/GlcNAcβ3)GlcNAc/GalNAc/GalαSer/Thr and glycopeptides and glycopeptide analogs comprising the O-glycan sequences. Even sequences lacking the non-reducing end GlcNAc may have some activity. Based on this all the other Helicobacter pylori binding oligosaccharide sequences (OS) and especially the trisaccharide epitopes are also especially preferred when linked from the reducing end to form structures OSβ6Gal(NAc)₀₋₁ or OSβ6Glc(NAc)₀₋₁ or OSβ6Gal(NAc)₀₋₁Ser/Thr or OSβ6Glc(NAc)₀₋₁αSer/Thr. The Ser or Thr-compounds or analogue thereof or the reducing oligosaccharides are also preferred when linked to a polyvalent carrier. The reducing oligosaccharides can be reductively linked to the polyvalent carrier.

The uronic acid comprising oligosaccharide sequences and the β6-linked oligosarccharides are also preferred in compositions and polysaccharide conjugates according to the present invention.

The present invention is further illustrated in examples, which in no way are intended to limit the scope of the invention:

### EXAMPLES

Materials and Methods

Materials—TLC silica gel 60 (aluminum) plates were from Merck (Darmstadt, Germany). All investigated glycosphingolipids were obtained in our laboratory. β-Galactosidase (Escherichia coli) was purchased from Boehringer Mannheim (Germany), Ham's F12 medium from Gibco (U.K.), ³⁵S-methionine from Amersham (U.K.) and FCS (fetal calf serum) was from Sera-Lab (England). β4-Galactosidase (Streptococcus pneumoniae), β-N-acetylhexosaminidase (Streptococcus pneumoniae) and sialidase (Arthrobacter ureafacins) were from Oxford GlycoSystems (Abington, U.K.). The clinical isolates of Helicobacter pylori (strains 002 and 032) obtained from patients with gastritis and duodenal ulcer, respectively, were a generous gift from Dr. D. Danielsson, Örebro Medical Center, Sweden. Type strains 17874 and 17875 were from Culture Collection, University of Göteborg (CCUG). Polysaccharide from Zooglea ramigera was from Sigma (St. Louis, USA), it was soluted in water to concentration 0.6 mg/ml. Hyaluronic acid (prod no 385 908) and chondroitin sulphate A (prod. no 230 687) were from Calbiochem (La Jolla, Calif., USA) and were used as 3 mg/ml water solutions. Chitosan average (low molecular weight) was from Fluka (Buchs, Switzerland). The citosan was soluted in concentration 3 mg/ml in 50% acetic acid by warming (about 20 min, 75 degrees Celsius) and sonication the acetic acid solution was stored at −20 degrees C., part of the solution was neutralized by drying in vacuum centrifige for 2.51 h, and soluting the filmrlike residue back to original volume of 200 μl PH was between 6.0-6.5). In spots C10 the solution with 50% acetic acid was used and extra acid was removed when the plate was allowed to dry overnight at room temperature. Starburst™ dendrimer with 64 primary amine groups was from Aldrich ( ) and used in concentration 2 mg/ml.

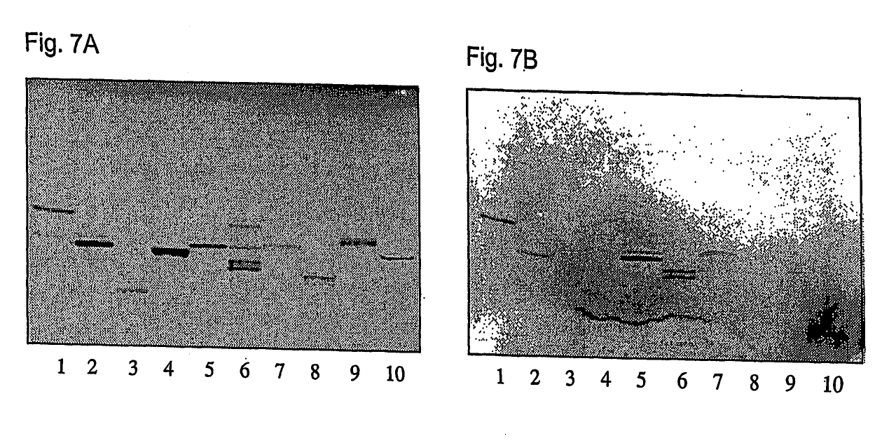

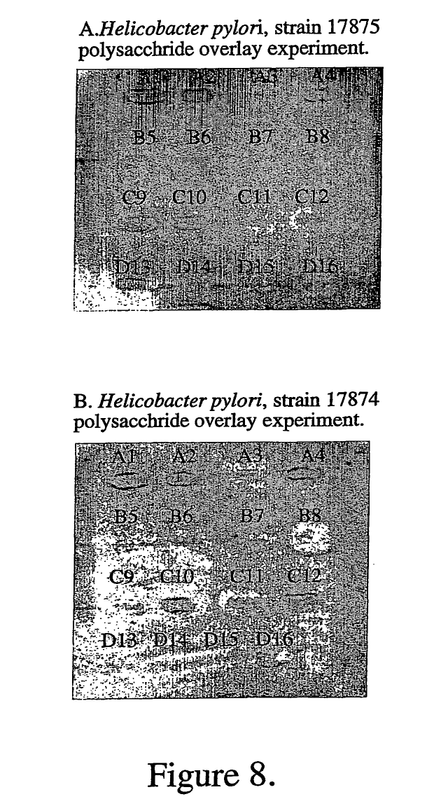

Glycosphingolipids. The pure glycosphingolipids of experiment shown in FIG. 7 were prepared from total acid or non-acid fractions from the sources listed in Table 2 as described in (Karlsson, 1987). In general, individual glycosphingolipids were obtained by acetylation (Handa, 1963) of the total glycosphingolipid fractions and separated by repeated silicic acid column chromatography, and subsequently characterized structurally by mass spectrometry (Samuelsson et al., 1990), NrM (Falk et al., 1979a,b,c; Koerner Jr et al., 1983) and degradative procedures (Yang and Hakomori, 1971; Stellner et al., 1973). Glycolipids derived from rabbit thymys are described below.

Purification of glycolipids. Acid glycosphingolipids were isolated from 1000 g acetone powder of rabbit thymus (Pel-Freeze Biologicals inc., North Arkansas, Ark. US). The acetone powder was extracted in a Soxhlet apparatus with chroloroform/methanol 2/1 (vol/vol unless otherwise stated) for 24 h followed by chloroform/methanol/water 8/1/1 for 36 h. The extracted lipids, 240 g, were subjected to Folch separation (Folch et al., 1957) and the collected hydrophllic phase to ion-exchange gel chromatography on DE23 cellulose (DEAE, Whatman, Maidstone, UK). These isolation steps gave 2.5 g of acid glycosphingolipids. The gangliosides were separated according to number of sialic acids by ion-exchange gel with open-tubular chromatographyon a glass column (50 mm i.d). The column was connected to an BPLC pump producing a concave gradient (pre-programmed gradient no 4, System Gold Chromatographic Software, Beckman Instruments Inc., CA, USA) starting with methanol and ending with 0.5 M CH₃COONH₄ in methanol. The flow rate was 4 ml/min and 200 fractions with 8 ml in each were collected. 300-400 mg of ganglioside mixture was applied at a time to 500 g of DEAE Sepharose, (CL6, Pharmacia, Uppsala, Sweden, bed height approx. 130 mm). The monosialylated gangliosides were further separated by HPLC on a silica column, 300 mm ×22 mm id., 120 Å pore size, 10 μm particle size (SH-044-10, Yamamura Ltd., Kyoto, Japan). Approximately 150 mg of monosialylated gangliosides were applied at time and a streight eluting gradient was used (chloroform/methanol/water from 60/35/8 to 10/103, 4 ml/min, 240 fractions).

Partial acid hydrolysis—Desialylation of gangliosides was performed in 1.5% CH₃COOH in water at 100° C. after which the material was neutralized with NaOH and dried under nitrogen. For partial degradation of the carbohydrate backbone the glycolipid was hydrolyzed in 0.5M HCl for 7 min in a boiling water bath The material was then neutralized and partitioned in C/M/H₂O, (8:4:3, v/v)². The lower phase was collected, evaporated under nitrogen and the recovered glycolipids were used for analysis.

Preparation of pentaglycosyl ceramide from hexaglycosyl ceramide by enzyme hydrolysis—Hexaglycosylceramide (structure 2, Table 1) obtained from heptaglycosylceramide (4 mg, from rabbit thymys) (structure 1, Table 1) by acidic desialylation (see above) was redissolved in C/M (2:1) and applied to a small silica gel column (0.4×5 cm). The column was eluted with C/M/H₂O (60:35:8, v/v). Fractions of about 0.2 ml were collected and tested for the presence of carbohydrates. The recovered hexaglycosylceramide (2.0 mg) was dissolved in 1.5 ml of 0.1 M potassium phosphate buffer, pH 7.2, containing sodium taurodeoxycholate (1.5 mg/ml), MgCl₂ (0.001M) and β-galactosidase (E. coli, 500 U when assayed with 2-nitrophenyl-β-D-galactoside as a substrate), and the sample was incubated overnight at 37° C. The material was next partitioned in C/M/H₂O (10:5:3) and the glycolipid contained in the lower phase was purified using silica gel chromatography (0.4×5 cm columns) as described above for hexaglycosylceramide. To remove all contaminating detergent the chromatography was repeated twice. The final recovery of pentaglycosylceramide was 0.7 mg.

Endoglycoceramidase digestion of glycolipids (Ito and Yamagata, 1989)—The reaction mixture contained 200 μg of glycolipid, 80 μg of sodium taurodeoxycholate and 0.8 mU of enzyme in 160 μl of 50 mM acetate buffer, pH 6.0. The sample was incubated overnight at 37° C., after which water (140 μl) and C/M, (2:1, by vol., 1500 μl) were added, and the sample was shaken and centrifuged. The upper phase was dried under nitrogen, redissolved in a small volume of water and desalted on a Sephadex G-25 column (0.4×10 cm), which had been equilibrated in H₂O, and eluted with water. Fractions of about 0.1 ml were collected and tested for the presence of sugars.

Permethylation of saccharides—Permethylation was performed according to Larson et al., 1987. Sodium hydroxide was added to samples before methyl iodide as suggested by Needs and Selvendran 1993. In some experiments the saccharides were reduced with NaBH₄ before methylation. In this case the amount of methyl iodide was increased to a final proportion of DMSO (dimethylsulfoxide)/methyl iodide of 1:1 (Hansson and Karlsson, 1990).

Gas chromatography/mass spectrometry—Gas chromatography was carried out on a Hewlett-Packard 5890A Series II gas chromatograph equipped with an on-column injector and a flame ionization detector. Permethylated oligosaccharides were analyzed on a fused silica capillary column (Fluka, 11 m×0.25 mm i.d.) coated with cross-linked PS264 (film thickness 0.03 μm). The sample was dissolved in ethyl acetate and injected on-column at 80° C. The temperature was programmed from 80° C. to 390° C. at a rate of 10° C./min with a 2 min. hold at the upper temperature. Gas chromatography-mass spectrometry of the permethylated oligosaccharides was performed on a Hewlett-Packard 5890A Series II gas chromatograph interfaced to a JEOL SX-102 mass spectrometer (Hansson and Karlsson, 1990). FAB-MS analyses were performed on a JEOL SX-102 mass spectrometer. Negative FAB spectra were produced using Xe atom bombardment (10 kV) and triethanolamine as matrix.

NMR spectroscopy—Proton NMR spectra were recorded at 11.75 T on a Jeol Alpha 500 (Jeol, Tokyo, Japan) spectrometer. Samples were deuterium exchanged before analysis and spectra were then recorded at 30 ° C. with a digital resolution of 0.35 Hz/pt Chemical shifts are given relative to TMS (tetramethylsilane) using the internal solvent signal.

Analyffcal enzymatic tests—Oxford GlycoSystems enzymatic tests were performed according to the manufacturer's recommendations except that Triton X-100 was added to each incubation mixture to final concentration of 0.3%. When a mixture of sialidase and β4-galactosidase were taken for digestion the incubation buffer from β4-galactosidase kit was used. If β-hexosaminidase was present in the digestion mixture the buffer from this enzyme kit was employed. The enzyme concentrations in the incubation mixtures were: 80 mU/ml for Hexβ4HexNAc-galactosidase (S. pneumoniae), 120 mU/ml for β-N-Acetylhexosaminidase (S. pneumoniae) and 1 U/ml for sialidase (Arthrobacter ureafaciens) The concentration of substrate was about 20 μ/M. Enzymatic digestion was performed overnight at 37° C. After digestion the samples were dried and desalted using small columns of Sephadex G-25 (Wells and Dittmer, 1963), 0.3 g, equilibrated in C/M/H₂O, (60:30:4.5, by vol.). Each sample was applied on the column in 2 ml of the same solvent and eluted with 2.5 ml of C/M/H₂O, (60:30:4.5) and 2.5 ml of C/M, (2:1). Application and washing solutions were collected and evaporated under nitrogen.

Other analytical methods—Hexose was determined according to Dubois et al. 1956.

De-N-acylation. Conversion of the acetamido moiety of GlcNAc/GalNAc residues into an amine was accomplished by treating various glycosphingolipids with anhydrous hydrazine as described previously (Ångström et al., 1998).

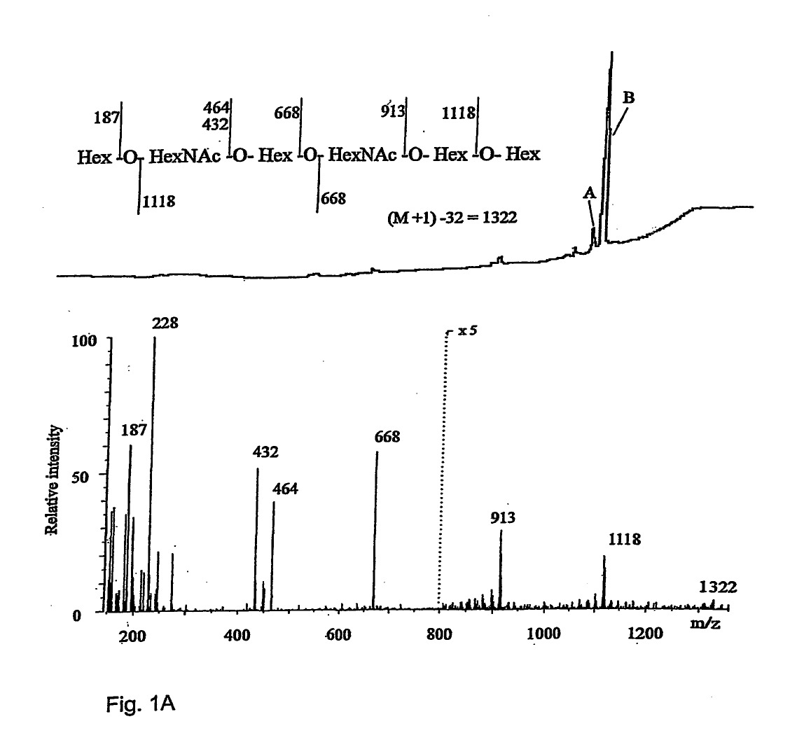

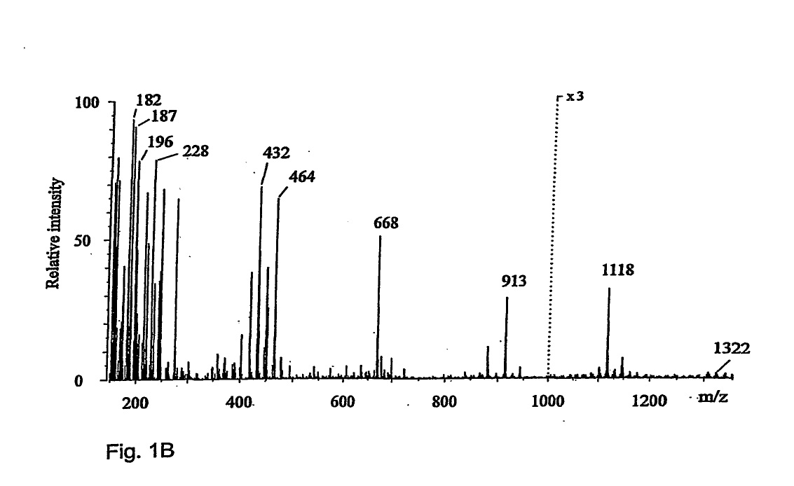

Bacterial growth. The Helicobacter pylori strains were stored at −80 ° C. in tryptic soy broth containing 15% glycerol (by volume). The bacteria were initially cultured on on GAB-CAMP agar (Soltesz et al., 1988) under humid (98%) microaerophilic conditions (O₂: 5-7%, CO₂: 8-10% and N₂: 83-87%) at 37° C. for 48-72 h. For labeling colonies were inoculated on GAB-CAMP agar, except for the results presented in FIG. 1 where Brucella agar (Difco, Detroit, Mich.) was used instead, and 50 μCi³⁵S-methionine (Amersham, U.K.), diluted in 0.5 ml phosphate-buffered saline (PBS), pH 7.3, was sprinkled over the plates. After incubation for 12-24 h at 37° C. under microaerophilic conditions, the cells were scraped off, washed three times with PBS, and resuspended to 1×10⁸ CFU/ml in PBS. Alternatively, colonies were inoculated (1×10⁵ CFU/ml) in Ham's F12 (Gibco BRL, U.K), supplemented with 10% heat-inactivated fetal calf serum (Sera-Lab). For labeling, 50 μCi ³⁵S-methionine per 10 ml medium was added, and incubated with shaking under microaerophilic conditions for 24 h. Bacterial cells were harvested by centrifugation, and purity of the cultures and a low content of coccoid forms was ensured by phase-contrast microscopy. After two washes with PBS, the cells were resuspended to 1×10⁸ CFU/mil in PBS. Both labeling procedures resulted in suspensions with specific activities of approximately 1 cpm per 100 Helicobacter pylori organisms.

TLC bacterial overlay assay. Thin-layer chromatography was performed on glass- or aluminum-backed silica gel 60 HPTLC plates (Merck, Darmstadt, Germany) using chloroform/methanol/water 60:35:8 (by volume) as solvent system. Chemical detection was accomplished by anisaldehyde stainig (Waldi, 1962). The bacterial overlay assay was performed as described previously (Hansson et al., 1985). Glycosphingolipids (1-4 μg/lane, or as indicated in the figure legend) were chromatographed on aluminum-backed silica gel plates and thereafter treated with 0.3-0.5% polyisobutylmethaerylate in diethylether/n-hexane 1:3 (by volume) for 1 min, dried and subsequently soaked in PBS containing 2% bovine serum albumin and 0.1% Tween 20 for 2 h A suspension of radio-labeled bacteria (diluted in PBS to 1×10⁸ CFU/ml and 1-5×10⁶ cpm/ml) was sprinkled over the chromatograms and incubated for 2 h followed by repeated rinsings with PBS. After drying the chromatograms were exposed to XAR-5 X-ray films (Eastman Kodak Co., Rochester, N.Y., USA) for 12-100 h.

Results

The heptaglycosylceramide Neu5Gcα3Galβ4GlcNAcβ3Galβ4GlcNAcβ3Galβ4GlcβCer was purified from rabbit thymus by HPLC as described above. The structure was characterized by NMR and mass spectrometry (data not shown). The heptasaccharide ganglioside was bound by most Helicobacter pylori isolates (about 60) tested in the laboratory of the inventors.

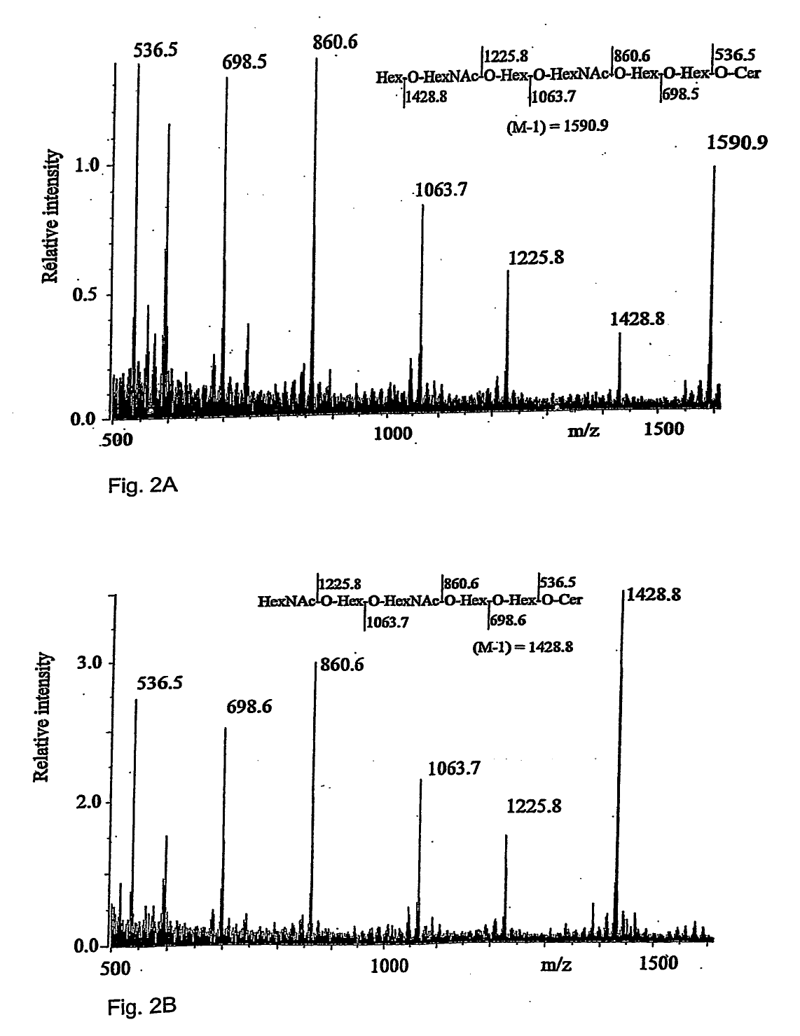

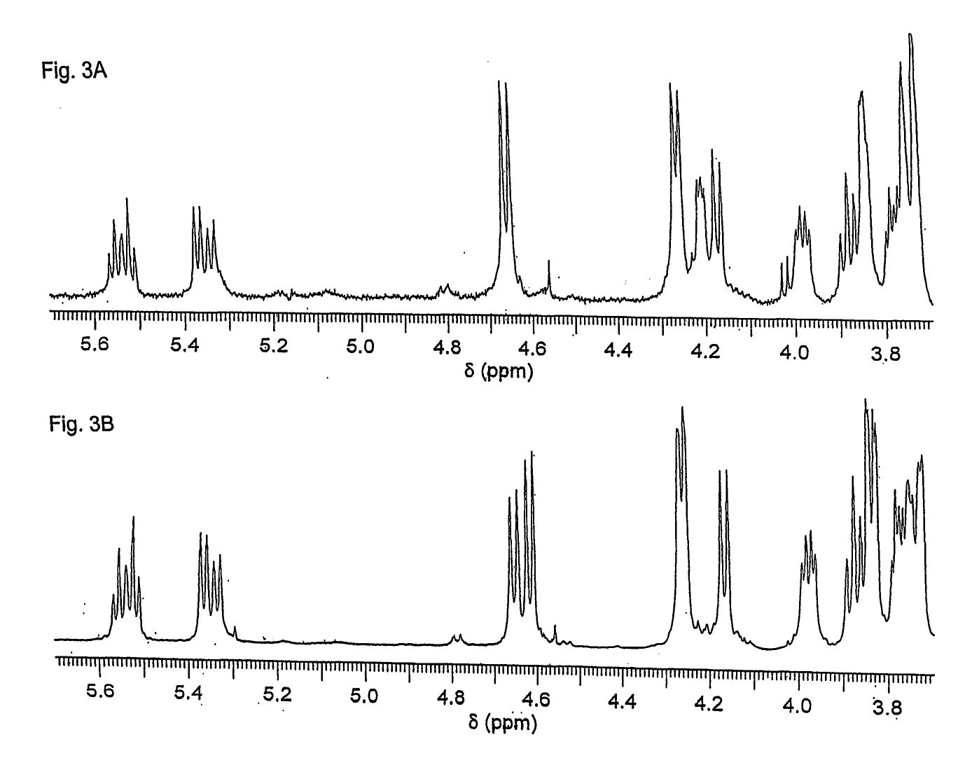

In order to detect possible minor isomeric components in the heptaglycosylceramide material, the ganglioside was desialylated, treated with endoglycoceramidase after which the released oligosaccharides were permethylated and analyzed by gas chromatography and EI/MS, (FIG. 1). Two saccharides were identified in the six-sugar region which showed the expected carbohydrate sequence of Hex-HexNAc-Hex-HexNAc-Hex-Hex, as confirmed by fragment ions at m/z 219, 464, 668, 913, and 1118. When carbohydrates were converted to alditols (by reduction with NaBH₄) before methylation distinct fragment ions at m/z 235, 684, and 1133 were found in addition to the previously listed ions (data not shown). The predominant saccharide, which accounted for more than 90% of the total material (peak B, FIG. 1), was characterized by a strong fragment ion at m/z 182 confirming the presence of β4GlcNAc (neolacto series, type 2 carbohydrate chain). The minor saccharide (peak A, FIG. 1) gave a spectrum typical for type-1 chain (lacto series) with a very weak fragment ion at m/z 182 and a strong fragment ion at m/z 228. The preparation also contained traces of other sugar-positive substances which might be 4- and 5-sugar-containing saccharides of the same series. Fucose-containing saccharides were not found in the mixture. The purity of the asialoganglioside was tested also by FAB/MS and NMR spectroscopy. The negative FAB/MS of the hexaglycosylceraide (FIG. 2, A) confirmed the predicted carbohydrate sequence and showed that the ceramides were composed mainly of sphingosine and C16:0 fatty acid (m/z 536.5). The NMR spectrum obtained of hexaglycosylceramide (FIG. 3, A) showed four major doublets in the anomeric region with β-couplings (J˜8 Hz). They had an intensity ratio of 2:2:1:1. The signals at 4.655 ppm (GlcNAcβ3), 4.256 ppm (internal Galβ4), 4.203 ppm (terminal Galβ4) and 4.166 ppm (Glcβ) were in agreement with results previously published for nLcOse₆-Cer (Clausen et al., 1986). There was also a small doublet at 4.804 ppm, which together with a small methyl signal at 1.81 ppm (seen as a shoulder on the large type 2 methyl resonance) indicated the presence of a small fraction of type 1 chain. Due to the overlap in the 4.15 to 4.25 ppm region the position and distribution of this type 1 linkage could not be determined. The total amount of type 1 linkage was roughly 10%. As the amount of type 1 chain in the pentaglycosylceramide obtained from hexaglycosylceramide by β-galacosidase digestion also was approximately 5% (FIG. 3, B) it seems likely that the type 1 linkage was evenly distributed between the internal and external parts of the saccharide chain, i.e. 5% of the glycolipids could be typel-typel.

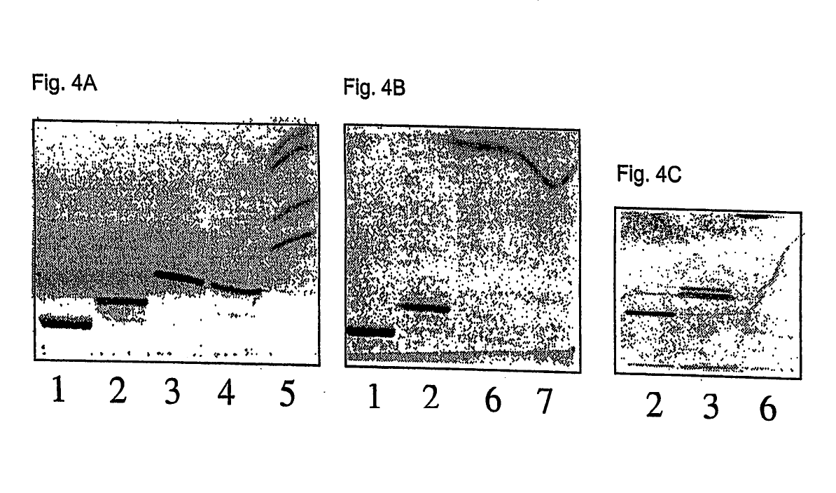

To find out if the binding activity of the glycolipid was associated with the predominant neolacto (type 2) structure the asialo-glycolipid was treated with β4-galactosidase and β-hexosaminidase, and the products were investigated by TLC and by overlay tests (FIG. 4). As expected, the first enzyme converted the hexaglycosylceramide to a pentaglycosylceramide (A, lane 3) and the mixture of the two enzymes degraded the material to lactosylceramide (B, lane 6). According to visual evaluation of the TLC plates both reactions were complete or almost complete. The same results were obtained for sialidase- and acid-treated material. The β4galactosidase degradation of hexaglycosylceramide was accompanied by disappearance of the Helicobacter pylori binding. activity in the region of this glycolipid on TLC plates with simultaneous appearance of a strong activity in the region of pentaglycosylceramides (C, lane 3). Further enzymatic degradation of the pentaglycosylceramide resulted in the disappearance of binding activity in this region. Appearance of binding activity in the four-sugar region was not observed. The sensitivity of the chemical staining of TLC plates is too low to allow trace substances to be observed.

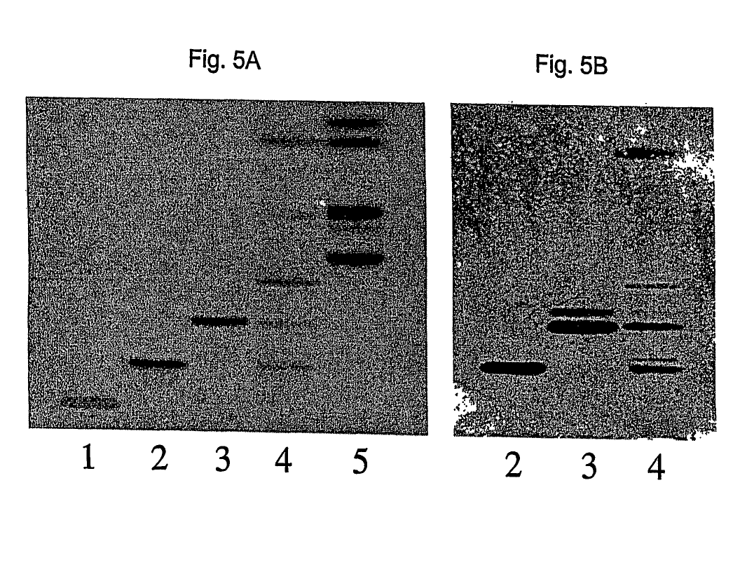

In a separate experiment the parent ganglioside was subjected to partial acid degradation and the released glycolipids were investigated for Helicobacter pylori binding activity. FIG. 5 shows TLC of the hydrolyzate (A) and the corresonding autoradiogram (B) after overlay of the hydrolyzate with ³⁵S-labeled Helicobacter pylori. Glycolipids located in the regions of hexa-, penta-, tetra- and diglycosylceramides displayed binding activity, whereas triglycosylceramide was inactive.

The binding of the hexa-, penta-, tetraglycosylceramides were similar when tested with at least three Helicobacter pylori strains (17875, 002 and 032).

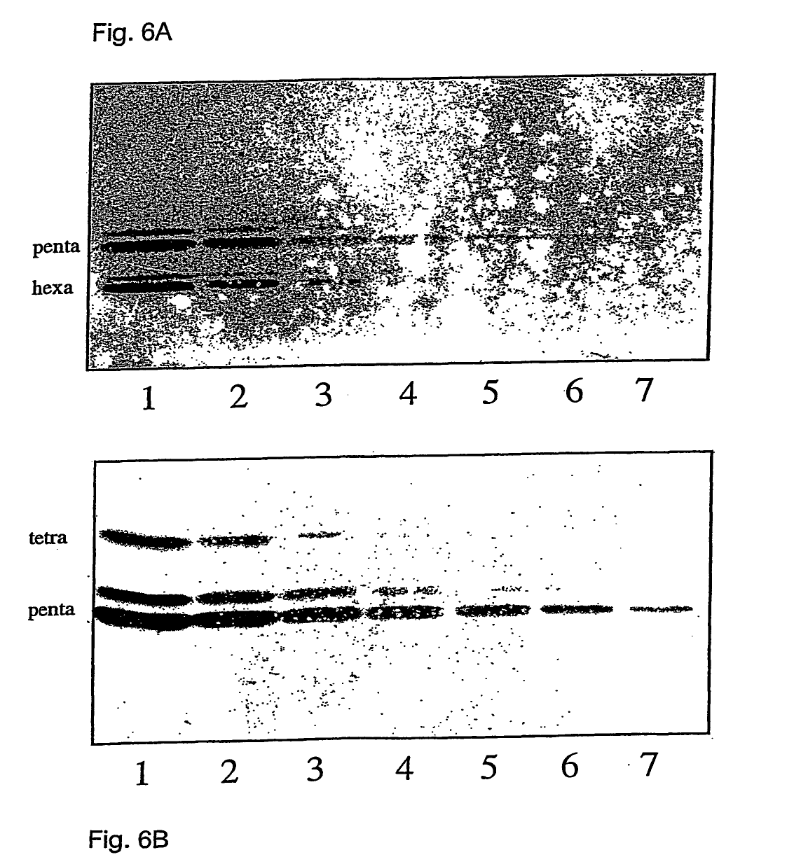

The strongly binding pentaglycosylceramide produced after detachment of the terminal galactose from hexaglycosylceramide and purification by silica gel chromatography was investigated in greater detail. The negative ion FAB/MS spectrum of this glycolipid confirmed a carbohydrate sequence of HexNAc-Hex-HexNAc-Hex-Hex- and showed the same ceramide composition as the hexaglycosylceraride (FIG. 2, B). The proton NMR spectrum obtained for the pentaglycosylceramide (FIG. 3, B) had five major β-doublets in the anomeric region: at 4.653 ppm (internal GlcNAcβ3), 4.615 ppm (terminal GlcNAcβ33), 4.261 ppm (double intensity, internal Galβ4), 4.166 (Glcβ3), consistent with GlcNAcβ3Galβ4GlcNAcβ3Galβ34GlcβCer and also in perfect agreement with the six sugar compound having been stripped of its terminal Galβ. There is also a small β-doublet at 4.787 ppm corresponding to 3-substituted GlcNAcβ (type 1 chain). The expected methyl signal was also seen as a shoulder on a much larger methyl signal at 1.82 ppm, but overlap prohibits quantitation of these signals. From the integral of the anomeric proton it can be calculated that 6% of the glycolipid contained type 1 chain. Thus the relative proportion of type 2 and type 1 carbohydrate chains was similar to that of the six sugar glycolipid. The two spots visible on TLC plates both in the hexa- and pentaglycosyl fractions reflected a ceramide heterogeneity rather than differences in sugar chain composition as judged by their susceptibility to β4-galactosidase. The upper penta-region spot appeared both after unselective hydrolysis of the asialoganglioside and selective splitting of linlked galactose from the asialo product. Furthermore, when hexaglycosylceramide with a high content of the upper chromatographic subfraction was degraded by β4-galactosidase and β-hexosaminidase the resulting lactosylceramide gave two distinct chromatographic bands. Chromatographically homogenous hexaglycosylceramide resulted in only one lactosylceramide band. Both upper and lower subfractions in the penta-region were highly active as shown by overlay tests. Glycosphingolipids of the neolacto series with 6, 5 and 4 sugars (structures 2, 4 and 5, Table I) were examined by semi-quantitative tests using the TLC overlay procedure. The glycolipids were applied on silica gel plates in series of dilutions and their binding to Helicobacter pylori was evaluated visually after overlay with labeled bacteria and autoradiography ( FIG. 6). The most active species was pentaglycosylceramide, which gave a positive response on TLC plates in amounts down to 0.039 nmol/spot (mean value calculated from 7 experiments, standard deviation δ₁=0.016 nmol). Hexa- and tetraglycosylceramides bound Helicobacter pylori in amounts of c:a 0.2 and 0.3 nmoles of glycolipid/spot, respectively. The binding of Helicobacter pylori to higher glycolipids of the investigated series was highly reproducible. The binding frequency for Helicobacter pylori, strain 032, recorded for pentaglycosyl- and hexaglycosylceramides was ˜90% (total number of plates was about 100).

Binding assays revealing the isoreceptors and specificity of the binding (FIG. 7.) In addition to the seven-sugar glycosphingolipid from rabbit thymus having a neolacto core, Neu5Gcα3Galβ4GlcNAcβ3Galβ4GcNAcβ3Galβ4GlcβCer, and tetra- to hexaglycosylceramides derived thereof, the binding specificity could involve other glycolipids from the neolacto senes.

The binding of Helicobacter pylori (strain 032) to purified glycosphingolipids separated on thin-layer plates using the overlay assay is shown in FIG. 7. These results together with those from an additional number of purified glycosphingolinids are summarized in Table 2. The binding of Helicobacter pylori to neolactotetraosylceramide (lane 1) and the five- and six-sugar glycosphingolipids (lanes 5 and 6) derived from Neu5Gcα3Galβ4GlcNAcβ3Galβ4GlcNAcβ3Galβ4GlcβCer is identical to results above. Unexpectedly, however, binding was also found for GalNAcβ3Galβ4GlcNAcβ3Galβ4GlcβCer (x₂ glycosphingolipid, lane 7) and the de-filcosylated A62 glycosphingolipid GalNAcα3Galβ4GlcNAcβ3Galβ4GcopCer (no. 12, Table 2). Togethtr with the finding that Galac3Galβ4GlcNAcβ3Galβ4GlcβCer (B5 glycosphingolipid, lane 2) also is binding-active, these results suggest the possibility of cross-binding rather than the presence of multiple adhesins specific for each of these glycosphingolipids (see below). Furthermore, the only extension of the different five-sugar-containing glycosphingolipids just mentioned that was tolerated by the bacterial adhesin was Galβ4 to the thymus-derived GlcNAcβ3-terminated compound (lane 6). Other elongated structures, as the Neu5Ac-x₂ (lane 8) and GalNAcβ3-B5 (no. 25, Table 2), were thus all found to be non-binding. It may be further noticed that the acetamido group of the internal GlcNAcβ3 in B5 is essential for binding since de-N-acylation of this moiety by treatment with anhydrous hydrazine leads to complete loss of binding (lane 3) as is the case also when neolactotetraosylceramide is similarly treated (no. 6, Table 2).

As mentioned above, the fact that there are four binding-active five-sugar glycosphingolipids (nos. 10-13, Table 2), all having a neolacto core, suggests that cross-binding to the same adhesin site may be the reason behind these observations.

Delineation of the neolacto binding epitope. The relative binding strength of the structures obtained by chemical and enzymatic degradation of the rabbit thymus seven-sugar compound (nos. 1, 5, 10, and 21, Table 2) suggest that the three-sugar sequence GlcNAcβ3Galβ4GlcNAcβ3 may constitute the minimal binding sequence. Thus, in the six-sugar compound an inhibitory effect from the terminal GalP4 is expected, whereas for neolactotetraosylceramide lack of a terminal GlcNAcβ3 reduces the binding strength since only two out of three sugars in the epitope are present. The essentiality of the internal GlcNAcβ3 is clearly shown by the loss of bacterial binding both to neolactotetraosylceramide and B5 following de-N-acylation of the acetamido group to an amine (nos. 6 and 14, Table 2). This non-binding may occur either by loss of a favorable interaction between the adhesin and the acetamido moiety and/or altered conformational preferences of these glycosphingolipids. However, it is difficult to envision a situation where an altered orientation of the internal Galβ4 would sterically hinder access to the binding epitope. Thus, having established that the mirnimal binding sequence must encompass the GlcNAcβ3Galβ4GlcNAcβ3 sequence it is now easy to rationalize the absence of binding for P₁, H5-2 and the two sialylparagloboside structures (nos. 15, 18-20, Table 2) since these extensions interfere directly with the proposed binding epitope. Also the glycosphingolipid from bovine buttermilk (Teneberg et al., 1994), which has a β6-linked branch of Galβ4GlcNAcβ attached to the internal Galβ4 of neolactotetraosylceramde (no. 26, Table 2), is non-binding due to blocked access to the binding epitope.

Elongation of the different binding-active five-sugar sequences in Table 2 shows that only addition of Galβ4 to the thymus-derived structure is tolerated, in accordance with the observation that the 4-OH position may be either equatorial or axial, but with an ensuing loss of binding affinity due to steric interference. Addition of either Neu5Acα3 to x₂ or GalNAcβ3 to B5 thus results in complete loss of binding (nos. 24 and 25, Table 2). It is farther seen that the negative influence of a Fucα2 unit as in H5-2 is confirmed by the non-binding of Helicobacter pylori both to A6-2 and B6-2 (nos. 22 and 23, Table 2). Concerning the elongated structure (no. 28, Table 2), terminated by the same trisaccharide found in B5, it must, as in B5, be this terminal trisaccharide that is responsible for the observed binding although a second internal binding epitope also is present. However, binding to the internal epitope can most likely be excluded since the penultimate Galβ4 would be expected to reduce the binding strength similarly to what is observed for the six-sugar compound from rabbit thymus relative to the five-sugar structure. It should also be pointed out that the sialic acid residue of the seven-sugar compound from rabbit thymus does not have an influence on the binding for the bacterial strains used in this study and must consequently be outside the epitope area Whether sialic acid-dependent or -independent binding of Helicobacter pylori is obtained or not depends, however, both on the type of strain and growth conditions (Miller-Podraza etal., 1996,1997a,b).

To summarize, the binding epitope of the neolacto series of glycosphingolipids has to involve the three-sugar sequence GlcNAcβ3Galβ34GlcNAcβ3 in order to obtain maximal activity. From a comparison of the binding pattern of the potential isoreceptors used in this study it can be deduced, that nearly all of this trisaccharide is important for binding to occur, excepting the acetamido group of the terminal GlcNAcβ3 and the 4-OH on the same residue, which are non-crucial. Motecular modeling results confiring the binding specificity are to be published separately.

### Example about Use of β83Galactosidase to Produce a Receptor Analog.

Hexasaccharide Galβ3GlcNAcβ3Galβ4GlcNAcβ3Galβ4Glc (1 mg, from Dextra labs, UK)) was treated with with 400 mU P3/6-galactosidase (Calbiochem, Calif., USA) overnight as suggested by the producer. 0.6 mg of pentasaccharide was obtained after HPLC-purcation steps, the saccharide was more than 98% pure when analyzed by mass spectrometry. Part of the pentasaccharide and maltoheptaose (Sigma, Saint Louis, USA) were reductively aminated with 4-hexadecylamnie (abbreviation HDA, from Aldrich, Stockholm, Sweden) by cyanoborohydride (Halina Miller-Podraza, to be published later). The products were characterized by mass spectrometry and were confirmed to be GlcNAcβ3Galβ4GlcNAcβ3Galβ4Glc(red)-HDA and maltoheptaose(red)-HDA [where “(red)-” means the amine linkage structure formed by reductive amination from the reducing end glucoses of the saccharides and amine group of the hexadecylaniline (HDA)]. The compound GlcNAcβ3Galβ4GlcNAcβ3Galβ4Glc(red)-HDA had observable binding activity with regard to Helicobacter pylori in TLC overlay assy described above while the maltoheptaose/red)-HDA was totally inactive. The example shows that the tetrasaccharide GlcNAcβ3Galβ4GlcNAcβ3Gal is a structure binding to Helicobacter pylori. The reducing end Glc-residue is probably not needed for the binding because the reduction destroys the pyranose ring structure of the Glc-residue.

Examples of reactions leading to glycan receptors of pathogenic agents.

a) Neu5Gcα3Galβ4GlcNAcβ3Galβ4GlcNAcβ3Galβ4GlcβCer, Neu5Acα3Galβ4GlcNAcβ3Galβ4GlcβCer and Neu5Acα3Galβ3GalNAcβ4Galβ4GlcβCer are acid hydrolyzed by 0,1 M HCl and glycosphingolipids Galβ4GlcNAcβ3Galβ4GlcNAcβ3Galβ4GlcβCer, Galβ4GlcNAcβ3Galβ4GlcβCer and Galβ3GalNAcβ4Gal4GlcβCer, respectively, are formed.

b) Galβ4GlcNAcβ3Gal4GlcNAc3Galβ4GlcβCer GlcNAcβ3Galβ4GlcNAcβ3Galβ4GlcβCer is formed.

c) Galβ4GlcNAcβ3Galβ4GlcNAcβ3Galβ4GlcβCer is Galβ4GlcβCer is formed.

d) Galβ4(Fucα3)GlcNAcβ3Galβ4GlcβCer is and Galβ4GlcNAcβ3Galβ4GlcβCer is synhesized.

e) Human erytirocytes containing blood group B antigens Galα3(Fucα2)Galβ4GlcNAcβ-Galα3Galβ4GlcNAcβ-,

Examples of binding between Helicobacter pylori and polysaccharide compositions. The polysaccharide compositons were spotted on thin-layer plates and the overlay assay with radiolabelled bacteria was used as shown in FIG. 8. The polysaccharides used were polysaccharide from Zooglea ramigera, chitosan, hyaluronic acid, and chondroitin sulphate. Amine containing molecules such as chitosan and Starburst-dendrimer (64 primary amine groups) were known to bind strongly to TLC-plate and these were also used as minxtures of the acidic polysaccharide, which could be partially released during the experiment. The dendrimer served also as a control thatthe binding to chitosan would not be only unspecific charge dependent phenomenon. Two separate experiments were done with bacterial strains 17874 and 17875. Helicobacter pylori bound the polysaccharide from Zooglea ramigera in dose dependent manner (spots A1 and B5) and also as complex with chitosan (C9), with possible weak bind also with the dendrimer complex (8A, D13). Helicobacter pylori also bound to neutral chitosan acetate salt strongly and dose dependently (spots A2, B6, C10). In case of hyaluronic acid the binding was very weak except for chitosan complex, possibly part of the hyaluronic acid diffused from the spot weakening the binding. Clear but weaker binding than with the first two polysaccharides were observed with chondroitin sulphate also as chitosan complex (spots A4, C12), diffision of the spot might have weakened the result. There was no observable binding to the amine dendrimer under the experimental conditions.

TABLE 1
Structures of glycosphingolipids discussed in the application.
The designation is according to recommendations
of IUPAC-IUB Joint Commissions on Biochemical
Nomenclature (Lipids 1977 12, 455; Eur. J. Biochem.
1998 257, 293).

| Structure | Symbol |
|---|---|
| 1. NeuGcα3Galβ4GlcNAcβ3Galβ4GlcNAcβ3Galβ4GlcβCer | VI³NeuGc-nLcOse₆Cer |
| 2. Galβ4GlcNAcβ3Galβ4GlcNAcβ3Galβ4GlcβCer | nLcOse₆Cer |
| 3. Galβ3GlcNAcβ3Galβ3GlcNAcβ3Galβ4GlcβCer | LcOse₆Cer |
| 4. GlcNAcβ3Galβ4GlcNAcβ3Galβ4GlcβCer | nLcOse₅Cer |
| 5. Galβ4GlcNAcβ3Galβ4GlcβCer | nLcOse₄Cer |
| 6. Galβ3GlcNAcβ3Galβ4GlcβCer | LcOse₄Cer |

TABLE 2
Binding of Helicobacter pylori to glycosphingolipids
separated on thin-layer chromatograms.

| | | | H. pylori | | |
|---|---|---|---|---|---|
| No. | Trivial name | Glycosphingolipid structure^{a} | binding^{b} | Source^{c} | References |
| 1 | Lactotri | GlcNAcβ3Galβ4Glcβ1Cer | − | RT | (Miller-Prodraza et al., 2001) |
| 2 | GgO3 | GalNAcβ4Galβ4Glcβ1Cer | (+) | GPE | (Yamakawa, 1966) |
| 3 | GgO3 (de-N-acylated) | GalNH₂β4Galβ4Glcβ1Cer | − | GPE^{e} | (Ångström et al., 1998) |
| 4 | Le^{y}-6 | Fucα2Galβ4(Fucα3)GlcNAcβ3Galβ4Glcβ1Cer | − | DSI | (McKibbin et al., 1982) |
| 5 | Neolactotetra | Galβ4GlcNAcβ3Galβ4Glcβ1Cer | (+) | HE^{f} | |
| 6 | Neolactotetra | Galβ4GlcNH₂β3Galβ4Glcβ1Cer | − | HE^{e} | |
| | (de-N-acylated) | | | | |
| 7 | GgO4 | Galβ3GalNAcβ4Galβ4Glcβ1Cer | + | HB^{g} | |
| 8 | GgO4 (de-N-acylated) | Galβ3GalNH₂β4Galβ4Glcβ1Cer | − | HB^{e} | (Ångström et al., 1998) |
| 9 | Le^{x}-5 | Galβ4(Fucα3)GlcNAcβ3Galβ4Glcβ1Cer | − | DSI | (Teneberg et al., 1996) |
| 10 | | GlcNAcβ3Galβ4GlcNAcβ3Galβ4Glcβ1Cer | + | RT^{d} | (Miller-Podraza et al., 2001) |
| 11 | x₂ | GalNAcβ3Galβ4GlcNAcβ3Galβ4Glcβ1Cer | + | HE | (Teneberg et al., 1996; Thorn et |
| | | | | | al., 1992) |
| 12 | | GalNAcα3Galβ4GlcNAcβ3Galβ4Glcβ1Cer | + | HE^{h} | |
| 13 | B5 | Galα3Galβ4GlcNAcβ3Galβ4Glcβ1Cer | + | RE | (Eto et al., 1968) |
| 14 | B5 (de-N-acylated) | Galα3Galβ4GlcNH₂β3Galβ4Glcβ1Cer | − | RE^{e} | |
| 15 | P₁ | Galα4Galβ4GlcNAcβ3Galβ4Glcβ1Cer | − | HE | (Naiki et al., 1975) |
| 16 | H5-1 | Fucα2Galβ3GlcNAcβ3Galβ4Glcβ1Cer | − | HM | (Karlsson and Larson, 1981a) |
| 17 | Le^{b}-6 | Fucα2Galβ3(Fucα4)GlcNAcβ3Galβ4Glcβ1Cer | − | HM | (Karlsson and Larson, 1981b) |
| 18 | H5-2 | Fucα2Galβ4GlcNAcβ3Galβ4Glcβ1Cer | − | HE | (Koscielak et al., 1973) |
| 19 | NeuAcα3-SPG | NeuAcα3Galβ4GlcNAcβ3Galβ4Glcβ1Cer | − | HE | (Ledeen and Yu, 1978) |
| 20 | NeuAcα6-SPG | NeuAcα6Galβ4GlcNAcβ3Galβ4Glcβ1Cer | − | HM | (Nilsson et al., 1981) |
| 21 | | Galβ4GlcNAcβ3Galβ4GlcNAcβ3Galβ4Glcβ1Cer | (+) | RT^{d} | (Miller-Podraza et al., 2001) |
| 22 | A6-2 | GalNAcα3(Fucα2)Galβ4GlcNAcβ3Galβ4Glcβ1Cer | − | HE | (Laine et al., 1974) |
| 23 | B6-2 | Galα3(Fucα2)Galβ4GlcNAcβ3Galβ4Glcβ1Cer | − | HE | (Koscielak et al., 1973) |
| 24 | NeuAc-x₂ | NeuAcα3GalNAcβ3Galβ4GlcNAcβ3Galβ4Glcβ1Cer | − | HE | (Watanabe and Hakomori, 1979) |
| 25 | | GalNAcβ3Galα3Galβ4GlcNAcβ3Galβ4Glcβ1Cer | − | RCC | (Thurin et al., 1989) |
| 26 | | Galβ4GlcNAcβ6(Galβ4GlcNAcβ3)Galβ4Glcβ1Cer | − | BB | (Teneberg et al., 1994) |
| 27 | | NeuGcα3Galβ4GlcNAcβ3Galβ4GlcNAcβ3Galβ4Glcβ1Cer | (+) | RT | (Lanne et al., 2001) |
| 28 | | Galα3Galβ4GlcNAcβ3Galβ4GlcNAcβ3Galβ4Glcβ1Cer | + | RT | (Lanne et al., 2001) |
| 29 | A7-2 | GalNAcα3(Fucα2)Galβ4(Fucα3)GlcNAcβ3Galβ4Glcβ1Cer | − | DSI | (Falk et al., 1979c) |
| 30 | B7-2 | Galα3(Fucα2)Galβ4(Fucα3)GlcNAcβ3Galβ4Glcβ1Cer | − | HE | |

Footnotes to Table 2
^{a}The glycosphingolipid shorthand nomenclature follows recent recommendations (Nomenclature of glycoproteins, 1988).
^{b}The following abbreviations are used for the glycosphingolipid sources: RT, rabbit thymus; HE, human erythrocytes; RE, rabbit erythrocytes; HM, human meconium; RCC, rat colon carcinoma; BB, bovine buttermilk; DSI, dog small intestine.
^{c}Definition of binding strength is as follows: + denotes a significant darkening of the autoradiogram with 4 μg applied on the TLC plate, (+) indicates a weak to intermediate darkening while a minus sign signifies no binding.
^{d}Prepared from No. 27 by mild acid hydrolysis and No. 10 by subsequent treatment with β-galactosidase.
^{e}Glycosphingolipid Nos. 3, 6, 8 and 14 were prepared from Nos. 2, 5, 7 and 13, respectively, by treatment with anhydrous hydrazine.
^{f}Prepared from no. 19 by neuraminidase treatment.
^{g}Prepared by mild acid hydrolysis of GM1 ganglioside from human brain.
^{h}Prepared from No. 22 by incubation in 0.05 M HCl at 80° C. for 2 h.

References

Aggarwal, B. B., Eessalu, T. E. and Hass, P. E. (1985) Nature, 318, 665-667. Andersson, B., Porras, O., Halson, L.Å., Lagergård, T., and Svanborg-Edén, C. (1986) J. Inif. Dis. 153, 232-7

Ångström, J., Teneberg, S., Abul Milh, M., Larsson, T., Leonardsson, I., Olsson, B.-M., Appelmelk, B. J., Faller, G., Clayes, D., Kirchner, T., and Vandenbroucke-Grauls, C.M.J.B. (1998) Immol. Today 19, 296-299.

Ascencio, F., Fransson, L-Å. and Wadstrböm, T. (1993) J. Med. Microbiol., 38, 240-244. Avenaud, P., Marais, A., Monteiro, L., Le Bail, B., Biolac Saga, P., Balabaud, C., and Megraud, F. (2000) Cancer 89, 1431-1439.

Axon, A. T. R (1993) J. of Antimicrobial Chemotherapy, 32, 61-68.

Babior, B. M. (1978) N. Eng. J. Med., 298, 659-668.

Babu, Y. S., Chand, P., Bantia, S., Kotian, P., Dehghani, A., El-Kattan, Y., Lin, T.-H., Hutchison, T. L., Elliot, A. J., Parker, C. D., Ananth, S. L., Horn, L. L., Laver, G. W., and Montgomery, J. A. (2000) J. Med. Chem. 43, 3482-3486.

Barnes, W. A., Redo, S. F., Ecker, R. R., and Wenig, J. (1967) Am. J. Surg., 113, 27-31.

Blaser, M. J. (1992) Eur. J. Gastroenterol. Hepatol., 4 (suppl 1), 17-19.

Bock K., Breimer, M. E., Brignole, A., Hansson, G. C., Karlsson, K.-A., Larson, G., Leffiler, H., Samuelsson, B. E., Strömberg, N., Svanborg-Edén, C. and Thurin, J. (1985) J. Biol. Chem., 260, 8545-8551.

Borén, T., Falk, P., Roth, K. A., Larson, G. and Normark, S. (1993)Science, 262, 1892-1895.

Breg, J., Kroon-Batenburg, L. M. J., Strecker, G., Montreuil, J. and Vhiegenthart, F. G. (1989) Eur. J. Biochem., 178, 727-739.

Castronovo, V., Colin, C., Parent, B., Foidart, J.-E, Lambotte, R., and Mahieu, P. (1989) J. Natl. Cancer Inst. 81, 212-216.

Chmiela, M., Czwianianc, E., Wadström, T, Czkwianianc, E., and Rudnicka, W. (1997) Gut 40, 20-24.

Chmiela, M., Wadström, T., Folkesson, H., Planeta Malecka, I., Czkwianianc, E., Rechcinski, T., and Rudnicka, W. (1998) Immunol. Leff. 61, 119-125.

Claeys, D., Faller, G., Appelnelk, B. J., Negrini, R., and Kirchner, T. (1998) Gastroenterology 115, 340-347.

Clausen, H., Levery, S. B., Kannagi, R. and Hakomori, S.-i. (1986) J. Biol. Chem., 261, 1380-1387.

Correa, T. L., Fox, J., Fontham, E., Ruiz, b., Lin, Y., zaula, D., Taylor, N., Mackinley, D., deLina, E., Portilla, H., Zarama, G. (1990) Cancer 66, 596-574.

DeCross, A. J. and Marshall, B. J. (1993) Am. J. Med Sci., 306, 381-392.

Dooley, C. P. (1993) Curr. Opin. Gastroenterol., 9, 112-117.

Dubois, M., Gilles, K. A. Hamilton, J, Rebers, P. A. and Smith, F. (1956) Analytical Chemistry 28,350-356.

Dunn, B. E., Cohen, H. and Blaser, M. J. (1997) Clin. Microbiol. Rev., 10, 720-741.

Ellis, C. M., Lunseth, J. B., and nicoloff, D. M. (1970) Am. J. Surg., 119, 213-216.

Eto, T., Ichikawa, Y., Nishinura, K, Ando, S. and Yamakawa, T. (1968) J. Biochem. (Tokyo), 64, 205-213.

Evans, D. G., Evans Jr, D. J., Molds, J. J., and Graham, D. Y. (1988) Infect Immun., 56, 2896-06

Falk, K.-E., Karlsson, K.-A. and Samuelsson, B. E. (1979a) Arch Biochem. Biophys., 192, 164-176.

Falk, K.-B., Karlsson, K.-A. and Samuelsson, B. E. (1979b)Arch. Biochem. Biophys., 192, 177-190.

Falk, K.-E., Karlsson, K.-A. and Samuelsson, B. E. (1979c) Arch Biochem. Biophys., 192, 191-202.

Farsak, B., Yildirir, A., Akyön, Y., Pinar, A., Öc, M., Böke, E., Kes, S., Tökgozoglu, L. (2000) J. clin. Microbiol. 38, 4408-4411.

Folch, J., Lees, M., And Sloane-Stanley, G.H. (1957) J. Biol. Chem. 226, 497-509.

Franzen, L.-E. and Norberg, A. B. (1984) Carbohydr. Res. 128, 111-117.

Fuilda, M. N., Dell, A, Oates, J. E., Wu, P., Klock, J. C. and Fukuda, M. (1985) J. Biol. Chem., 260, 1067-1082.

Gubareva, L. V., Kaiser, L., Hayden, F. G. The Lancet (2000) 355, 827-835.

Handa, S. (1963) Jap. J. Ep. Med., 33, 347-360.

Hanisch, F.-G., Kodolvsky, U., and Borchard, F. (1993) Cancer Res. 53, 4791-4796.

Hansson, G. C., Karlsson, K.-A., Larson, G., Strömberg, N. and Thurin, J. (1985) Anal. Biochemn., 146, 158-163.

Hansson, G. C. and Karlsson, H. (1990) Methods Enzymol. 193, 733-738.

Hillyard, I. W., Doczi, J., and Kiernan, P. B. (1964) Proc. Soc. Exp. Biol. Med. 115, 1108-1112.

Huber, R. E. and Brockbank, R. L. (1987) Biochemistry 26, 1526-1531.

Hu, J., Stults, C. L., Holmes, E. H., and Macher, B. A. (1994) Glycobiology 4, 251-7.

Icatlo, F. C. jr., Kimura, N., Gushima, H., and Kodama, Y. (2000) Antimicrob. Agents Chemother. 44, 2492-2497.

Ikeda, F., Shuto, H., Saito, T., Fukai, T., and Tomita K. (1982) Eur. J. Biochem. 123, 437-445.

Ito, M., Ban, A, and Ishihara M. (2000) Japan. J. Pharmacol. 82, 218-225.

Ito, M. and Yamagata, T. (1989) Methods Enzymol., 179, 488-496.

Jassel, S. V., Ardill, J. E. S., Fillmore, D., Bamford, K. B., O'Connor, F. A., and Buchanan, K. D. Q. J. Med 92, 373-377.

Kannagi, R, Levine, P., Watanabe, K and Hakomori, S.-i. (1982b) Cancer Res., 42, 5249-5254.

Karisson, K.-A. (1987) Meth. Enzymol., 138, 212-220.

Karlsson, K.-A. (1989) Annu. Rev. Biochem., 58, 309-350.

Karlsson, K.-A. and Larsson, G. (1981a) J. Biol. Chem. 256, 3512-3524.

Karlsson, K.-A. and Larsson, G. (1981b) FEBS Lett., 128, 71-74.

Kerr, J. R., Al-Khattaf, A., Barson, A. J., and Burnie, J. P. (2000) Arch. Child. Dis., 83, 429-434.

Koemer Jr., T. A. W., Prestegard, J. H., Demou, P. C. and Yu, R. K. (1983) Biochemistry, 22, 2676-2687.

Kopitz, J., Mühl C., Ehnnann, V., Lehmann, C., and Cantz, D (1997) Eur. J Cell Biol. 73, 1-9.

Koscielak, J., Piasek, A., Gorniak, H., Gardas, A. and Gregor, A. (1973) Eur. J. Biochem., 37, 214-215.

Krivan, H. C., Clark, G. F., Smith, D. F., and Wilkins, T. D. (1986) Infect. Immun. 53, 573-581.

Kumar, V., Kessler, J., Scot M. E., Patwardhan, B. H., Tanenbaum, S. W., and Flashner, M. (1981) Carbohydrate Res., 123-130.

Laine, R. A., Stellner, K. and Hakomori, S.-i. (1974) Meth. Membr. Biol., 2, 205-244.

Larne, B., Miller-Podraza, H., Abul Milh, M., Teneberg, S., Uggla, L., Larsson, T., Leonardsson, I., Jovall, P.-Å., Bergström, J. and Karlsson, K.-A. (2000) manuscript in preparation

Larsen, R. D., Rivera-Marrero, C. A., Ernst, L. K., Cummings, R. D. and Lowe, J. B. (1990) J. Biol. Chem., 265, 7055-7061.

Larson, G., Karlsson, H., Hansson, G. C. and Pimlott, W. (1987) Carbohydr. Res., 161, 281-290

Ledeen, R. and Yu, R. K. (1978) Res. Methods Neurochem., 4, 371-410.

Lin, J.-T., Wang, J.-T., Wang, M.-S., Wu, M.-S. and Chen, C.-J. (1993) Hepato-Gastroenterol., 40, 596-599.

Lingwood, C. A., Huesca, M. and Kuksis, A. (1992) Infect. Immun., 60, 2470-2474.

Mayo, S. L., Olafsen, B. D. and Goddard III, W. A. (1990) J. Chem. Phys., 94, 8897-8909.

McGee, C. M., and Murray, D. R. (1986) Ann Botany 57, 179-.

Meyer, B. (1990) Topics Curr. Chem., 154, 141-208.

Mier-Podraza, H., Abul Milh, M., Bergström, J. and Karlsson, I-A. (1996) Glycoconj. J., 13, 453-460:

Miller-Podraza, H., Bergstrom, J., Abul Milh, M. and Karlsson, K.-A. (1997a)Glycoconj. J., 14, 467-471.

Miller-Podraza, H., Abul Milh, M., Teneberg, S. and Karlsson, K.-A. (1997b) Infect. Immun., 65, 2480-2482.

Miller-Podraza, H., Abul Milh, M., Ångström, J., Jovall. P.-Å., Wilhelmsson, U., Lanne, B., Karlsson, H., and Karlsson, K.-A. (2000) manuscript in preparation.

Mysore, J. V., Wiggingtqn, T., Simon, P. M., Zop, D., Heran-Ackah, L. M. and Dubois, A. (1999)Gastroenterology, 117, 1316-1325

Naild, K, Fong, J., Ledeen, R. and Marcus, D. M. (1975) Biochemistry, 14, 4831-4836.

Needs, P. W. and Selvendra R. R. (1993) Carbohydr. Res., 245, 1-10.

Nilsson, H.-O., Taneera, J., Castedal, M., Glatz, E., Olsson, R., and Wadstrom, T. (2000) J. Clin. Microbiol. 38, 1072-1076.

Nilsson, O., Måansson, J.-E., Tibblin, E. and Svennerholm, L. (1981)FEBS Lett., 133, 197-200.

Nomenclature of glycoproteins (1988) J. Biol. Chem., 262, 13-18.

Nomura, A. and Stemmermann, G. N. (1993) J. Gastroenterol. Hepatol., 8, 294-303.

Nyholm, P. G., Samuelsson, B. E., Breimer, M. and Pascher, I. (1989) J. Mol. Recog., 2, 103-113.

Nyholm, P.-G. and Pascher, I. (1993) Biochemistry, 32, 1225-1234.

Nöhle, U., Shukla, A. K., Schröder, C., Reuter, G., Schauer, R., Kamerling, J. P., Vliegenthart J. F. G. (1985) Eur. J. Biochem. 152, 459-463.

Ofek, I. and Sharon, N. (1988) Infect. Immun., 56, 539-547.

Pakodi, F., Abdel-Salam, O.M.E., Debraceni, A., and Mozsik, G. (2000) J. Physiol. (Paris), 94, 139-152.

Platt, F. M., Neises, G. R., Karlsson, G. B., Dwek, R. A., and Butters, T. D. (1994) J. Biol. Chem. 269, 27108-27114.

Parsonnet, J., Friedman, G. D., Vandersteen, D. P., Chang, Y., Vogelman, J. H., Orentreich, N. and Sibley, R. K. (1991) N. Engl. J. Med., 325, 1127-31.

Rappé, A. K. and Goddard III, W. A. (1991) J. Chem. Phys., 95, 3358-3363.

Rautelin, H., Blomberg, B., Jörnerot, G. and Danielsson, D. (1994a) Scand. J. Gastroenterol., 29, 128-132.

Rautelin, H., von Bonsdorff, C.-H., Blomberg, B. and Danielsson, D. (1994b) J. Clin. Pathol., 47, 667-669.

Rebora, R., Drago, F., and Parodi, A. (1995) Dermatology 191, 6-8.

Rudick, J., Finkelstein, J., Kark, A. E., Dreiling, D. A. (1968) Proc. Soc. Exp. Biol. Med. 127, 781-3

Saitoh, T., Natomi, H., Zhao, W., Okuzumi, K., Sugano, K, Iwamori, M. and Nagai, Y. (1991)FEBS Lett., 282, 385-387.

Samuelsson, B. E., Pimlott, W. and Karlsson, R.-A. (1990) Meth Enzymol., 193, 623-646.

Sasaki, K., Kurata-Miura, K., Ujita, M., Angata, K., Nakagawa, S., Sekine, S., Nishi, T., and Fukada, M. (1997) Proc. Natl. Acad. Sci. (USA) 94, 14294-14299.

Schreiner, E., and Zbiral, E. (1985) Carbohydrate Res., 216, 61-66.

Siebert, H.-C., Reuter, G., Schauer, R., von der Lieth, C.-W. and Dabrowsld, J. (1992) Biochemistry, 31, 6962-6971.

Simon, P. M., Goode, P. L., Mobasseri, A., and Zopf, D. (1997) Infect. Immun. 65, 750-757 Soltesz, V., Schalen, C. and Måardh, P. A. (1988) Proceedings of the Fourth International Workshop on Campylobacter Infections (Kaijser, B. and Falsen, E., eds.) pp. 433-436, Gotema, Kungalv, Sweden.

Steininger, H., Faller, G., Dewald, E., Brabletz, T., Jung, A., and Kirchner, T. (1998) Virchows Arch. 433, 13-18.

Stellner, K, Saito, H. and Hakomori, S.-i. (1973) Arch. Biochem. Biophys., 155, 464-472.

Stellner, K. and Hakomori, S.-i. (1974) J. Biol. Chem., 249, 1022-1025.

Stroud, M. R., Handa, K, Salyan, M. E. K., Ito, K, Levery, S. B., Hakomori, S.-i., Reinhold, B. B. and Reinhold, V. N. (1996) Biochemistry, 35, 758-769.

Sung, J., Russell, R. I., Neyomans, Chan, F. K., Chen, S., Fock, K., Goh, K. L., Kullavanijaya, P., Kinura, K., Lau, C., Louw, J., Sollano, J., Triadiafalopulos, G., Xiao, S.,

Brooks, P. (2000) J. Gastroenterol. Hepatol., 15, Suppl: G58-68.

Teneberg, S., Ångström, J., Jovall, P.-Å. and Karlsson, K-A. (1994) J. Biol. Chem., 269, 8554-8563.

Teneberg, S., Lönnroth, I., Torres Lopez, J. F., Galili, U., Ölwegåard Halvarsson, M., Ångström, J. and Karlsson, K-A. (1996) Glycobiology, 6, 599-609.

Teneberg, S., Abul Milh, M., Lanne, B., Jovall, P.-Å, Karlsson, H., Ångström, J., Ölwegard Halvarsson, M., Danielsson, D., Ljung, Å, Wadström, T., and Karlsson, K.-A. (2001) Manuscript in preparation.

Thorn, J. J., Levery, S. B., Salyan, M. E. K., Stroud, M. R., Cedegren, B., Nilsson, B., Hakomori, S.-I. and Clausen, H. (1992) Biochemistry, 31, 6509-6517.

Thurin, J., Brodin, T., Bechtel, B., Jovall, P.-Å., Karlsson, H., Strömberg, N., Teneberg, S., Sjögren, H. O. and Karlsson, K.-A. (1989) Biochim. Biophys. Acta, 1002, 267-272.

Waldi, D. (1962) in Dönnschicht-Chromatographie (Stabl, E., ed.) pp. 496-515, Springer-Verlag, Berlin.

Watanabe, K. and Hakomori, S.-i. (1979) Biochemistry, 18, 5502-5504.

Wells, M. E. and Dittmer, J. C. (1963) Biochemistry, 2, 1259-1263.

Winchester, B., et al. (1990) Biochem. J. 265, 277-.

Wotherspoon, A. C., Doglioni, C., Diss, T. C., Pan, L., Moschini, A., de Boni, M. and Isaacson, P. G. (1993) Lancet, 342, 575-577

Woynarowska, B., Wikiel, H., Sharma, M., Carpenter, N., Fleet, G. W., and Bednacki, R. J. (1992) Anticancer Res. 12, 161-6.

Yamakawa, T. (1966) Colloq. Ges. Physiol. Chem., 16, 87-111.

Yang, H.-j. and Hakomori, S.-i. (1971) J. Biol. Chem., 246, 1192-1200.

What is claimed:

1. A composition comprising a polysaccharide with Helicobacter pylori receptor activity, wherein said polysaccharide is chltosan, chondroitin, fucosylated chondroitin, hyaluronic acid or a microbial idolysaccharide, for use in the treatment or prophylaxis of any condition due to the presence of Helicobacter pylori.

2. The composition according to claim 1 further comprising an oligosaccharide receptor of Helicobacter pylori or an analogue or a derivative thereof

3. The composition according to claim 1 or 2 further comprising a gastric epithelium protecting compound.

4. The composition according to claim 2, wherein said oligosaccharide receptor is an oligosaccharide sequence according to the following formula
[Galβy]ₚ[Hex(NAc)ᵣα/βz]ₛGalβ4Glc(NAc)ᵤ (I)
wherein p, r, s, and u are each independently 0 or 1, and y is either linkage position 3 or 4, and z is either linkage position 3 or 4, and Hex is either Gal or Glc,
so that when p is 1 and y=3, then Hex is Galβ or Glcp and r=1, or p is 1 and y=4 then Hex is Glcβ and r=1, when p is 0 and z is 4, then Hex is Galβ and r is 1.

5. The composition according to any one of claims 1-3, wherein said polysaccharide is a conjugate comprising a polysaccharide and an oligosaccharide receptor of Helicobacter pylori or an analogue or a derivative thereof.

6. The composition according to claim 5 wherein said conjugate comprises an oligosaccharide sequence of claim 4 covalently bound to a polysaccharide via a spacer molecule.

7. The composition according to any one of claims 1-3, wherein said polysaccharide is a conjugate comprising an amine containing polysaccharide and an acidic polysaccharide or a fragment thereof.

8. The composition according to claim 7, wherein said amine containing polysaccharide is chitosan.

9. The composition according to claim 4, wherein said oligosaccharide sequence is selected from the group of:
Galβ4Glc, GalNAcβ4Galβ4GlcNAc,
GalNAcβ4Galβ4Glc, Galβ3GalNAcβ4GalαGlc,
Galβ3GlcNAc, Galβ4GlcNAc, Galα3Galβ4GlcNAc, Galβ3Galβ4GlcNAc
Galα3Galβ4Glc, Galβ3Galβ4Glc, GalNAcα3Galβ4GlcNAc,
GalNAcβ3Galβ4GlcNac, GlcGalβ3Galβ4GlcNAc,
Galβ3GlcNAcβ3Galβ4Glc(NAc)₀₋₁, Galβ4GlcNAcβ3Galβ4Glc(NAc)₀₋₁,
Glc(NAc)₀₋₁α3Galβ4Glc(NAc)₀₋₁, Glc(NAc)₀₋₁β3Galβ4Glc(NAc)₀₋₁ and
Galβ4Glc(NAc)₀₋₁β, Galβ3GlcNAcβ, Galα3Galβ4GlcNAcβ,
GalNAcαGalβ4GlcNAcβ, GalNAcβ3Galβ4GlcNAcβ, and
GlcNAcβ3 Galβ4GlcNAcβ.

10. The composition according to claim 1, wherein said oligosaccharide receptor is a Lewis b saccharide sequence, NeuNAcα3Gal or NeuNAcα6Gal.

11. The composition according to any one of the claims 1-6 or 9-10, wherein said oligosaccharide receptor of Helicobacter pylori is a part of a glycolipid or glycoprotein.

12. The composition according any one of the preceding claims, wherein said gastric epithelium protecting compound is an acidic or amine containing polysaccharide.

13. The composition according to claim 12, wherein said acidic polysaccharide is heparin, heparan sulphate, heparinoid, carrageenan, dextran sulphate, cellulose sulphate, starch sulphate, sulphated amylopectin, fucoidan or an acidic/glucuronic acid containing bacterial polysaccharide.

14. The composition according to claims 1-11, wherein said gastric epithelium protecting compound is a drug regulating gastric pH comprising a compound buffering or neutralizing gastric pH, a compound protecting gastric epithelium from gastric acid or a compound inhibiting proton pumps.

15. The composition according to claim 14, wherein said drug regulating gastric pH is omeprazole, esomeprazole, lansoprazole, a histamine receptor antagonist or sucralfate.

16. The composition according to claim 1-3, wherein said oligosaccharide receptor of Helicobacter pylori is covalently bound to said polysaccharide.

17. The composition according to any one of claims 1-11 comprising said polysaccharide and said oligosaccharide receptor.

18. The composition according to any one of claims 1-3 or 12-15 comprising said polysaccharide and said gastric epithelium protecting compound.

19. The composition according to claim 17 comprising chitosan and acidic polysaccharide or a fragment thereof.

20. A repeating polysaccharide substance comprising several terminal oligosaccharide sequence according to formula
[Galβ4]ₚHex(NAc)ᵣα/β3Galβ4Glc(NAc)ᵤ
wherein p, r and u are each independently 0 or 1, and Hex is either Gal or Glc, so that when p is 1 then Hex is Glcβ and r is 1.

21. The repeating polysaccharide substance according to claim 20, wherein p is 0 and the said polysaccharide is from a non-pathogenic bacterium or a lactic acid bacterium.

22. Use of the composition or substance of any one of claims 1-21 for the manufacture of a medicament for the treatment of any condition due to the presence of Helicobacter pylori.

23. The composition or substance according to any one of claims 1-21 for the treatment of chronic superficial gastritis, gastric ulcer, duodenal ulcer, non-Hodgkin lymphoma in human stomach, gastric adenocarcinoma, certain pancreatic, skin, liver, or heart diseases, non-steroidal anti-inflanmnatory drug related gastric disease, autoimmune gastric disease, pernicious anemia, gastric adenocarcinoma, MALT lymphoma, side effects of gastric disease inducing drugs and sudden infant death syndrome.

24. A nutritional additive, food-stuff or beverage containing the composition or substance according to any one of claims 1-21.

25. The nutritional additive according to claim 24 for use in infant food.

26. The composition or substance according to any one of claims 1-21 for pharmaceutical use.

27. A pharmaceutical composition comprising a drug inducing gastric disease as side effect and the composition or substance according to any one of claims 1-21.

28. The composition or substance according to any one of clains 1-21 for use in the protection of a gastric epithelium.

29. The composition or substance according to any one of claims 1-21 for binding of Helicobacter pylori.

30. A method for the treatment of a condition due to the presence of Helicobacter pylor, wherein a pharmaceutically effective amount of the composition or substance according to any one 1-21 or 28-29 is administered to a subject in need of such treatment.

31. Method of producing polysaccharides with Helicobacter pylori receptor activity by transferring a monosaccharide or several monosaccharides on a polysaccharide or removing a monosaccharide residue or residues using a glycosyltransferase enzyme so that a receptor active oligosaccharide sequence is formed on the polysaccharide.

32. The method according to claim 31, wherein said glycosyltransferase enzyme is a glycosidase, glycosyl transferase or transglycosylating enzyme.

33. The composition according to claim 1 or conjugate according to any of the claims 5-7 or substance according to claim 20 or 21, wherein said oligosaccharide receptor in any one of the claims 1, or 5-7 or terminal oligosaccharide in claim 20 or 21 is an oligosaccharide sequence according to the following formula
[Gal(A)_{q}(NAc)ᵣ/Glc(A)_{q}(NAc)ᵣα3/β3]ₛ[Galβ4GlcNAcβ3]ₜGalβ4Glc(NAc)ᵤ
wherein q, r, s, t, and u are each independently 0 or 1.

34. The composition according to claim 1 or conjugate according to any of the claims 5-7, wherein said oligosaccharide receptor is an oligosaccharide sequence according to the following formula
GalANAcβ3Galβ4GlcNAc, GalANAcα3Galβ4GlcNAc,GalAβ3Galβ4GlcNAc, GalAα3Galβ4GlcNAc, GalANAcβ3Galβ4Glc, GalANAcα3Galβ4Glc, GalAβ3Galβ4Glc, GalAα3Galβ4Glc, GlcANAcβ3 Galβ4GlcNAc, GlcANAcα3Galβ4GlcNAc,GlcAβ3 Galβ4GlcNAc, GlcAα3Galβ4GlcNAc, GlcANAcβ3Galβ4Glc, GlcANAcα3Galβ4Glc,GlcAβ3Galβ4Glc, GlcAα3Galβ4Glc.

35. The composition according to claim 1 or conjugate according to any of the claims 5-7, wherein said oligosaccharide receptor is an oligosaccharide sequence according to the following formula
OSβ6Hex(NAc)ₙ
wherein
n is 0 or 1
and OS is any Helicobacter pylori recepor oligosaccharide sequence according to the invention.

36. The composition according to claim 35 wherein OS is a trisaccharide epitope.

37. A composition comprising a polysaccharide with Helicobacter pylori receptor activity, wherein said polysaccharide is chitosan, chondroitin, chondroitin sulphate, fucosylated chondroitin, hyaluronic acid or a microbial polysaccharide or said polysaccharide is a conjugate comprising a polysaccharide and an oligosaccharide receptor of Helicobacter pylori or an analogue or a derivative thereof according to the following formula
[Galβ4]ₚ[Hex(NAc)ᵣα3]ₛGalβ4Glc(NAc)ᵤ
wherein p, r, s, and u are each independently 0 or 1, and Hex is either Gal or Glc,
so that at least s is 1 or u is 1,
p is 1 only when s is 1 and Hex is Glcβ and r is 1,
for use in the treatment or prophylaxis of any condition due to the presence of Helicobacter pylori.
